(19) United States
(12) Patent Application Publication
(10) Pub. No.: US 2009/0041839 A1
(43) Pub. Date: Feb. 12, 2009

(54) PHARMACEUTICAL COMPOSITIONS FOR THE TREATMENT OF PAIN

(76) Inventors: Martin W. Beasley, Cary, NC (US); David P. Hause, Carrboro, NC (US); David J. Reynolds, Durham, NC (US)

Correspondence Address: KING PHARMACEUTICALS, INC., 400 CROSSING BOULEVARD, BRIDGEWATER, NJ, 08807, US

(21) Appl. No.: 12/125,511

(22) Filed: May 22, 2008

Related U.S. Application Data
(60) Provisional application No. 60/939,665, filed on May 23, 2007.

(51) Int. Cl. A61K 9/48; A61K 31/381

(52) U.S. Cl. 424/452; 514/443

(57) ABSTRACT

The present invention provides pharmaceutical compositions comprising an allosteric adenosine A₁ receptor enhancer, such as T-62, in an oral dosage form and processes for the manufacture of such compositions and dosage forms. In another aspect, the present invention relates to a method of employing such dosage forms for the treatment of pain, including acute pain, e.g., postoperative pain, chronic pain, inflammatory pain, neuropathic pain and pain associated with migraine.

This application claims the benefit of U.S. Provisional Application No. 60/939,665 filed May 23, 2007, incorporated herein by reference in its entirety.

### FIELD OF THE INVENTION

The present invention relates to pharmaceutical compositions suitable for oral administration of allosteric adenosine A₁ receptor enhancers. In particular, the present invention provides oral dosage forms comprising an allosteric adenosine A₁ receptor enhancer, e.g., a 2-amino-3-aroylthiophene derivative, such as T-62, and at least one lipid excipient. The present invention also provides processes for the manufacture of such pharmaceutical compositions, and oral dosage forms thereof, and their use as medicaments for the treatment of pain.

### BACKGROUND OF THE INVENTION

Adenosine is an endogenous nucleoside present in all cell types of the body. It is endogenously formed and released into the extracellular space under physiological and pathophysiological conditions characterized by an increased oxygen demand/supply ratio. This means that the formation of adenosine is accelerated in conditions with increased high energy phosphate degradation. The biological actions of adenosine are mediated through specific adenosine receptors located on the cell surface of various cell types, including nerves. The hyper-reactive nerves increase adenosine release due to an increase in metabolic activity.

Adenosine A₁ receptors are widely distributed in most species and mediate diverse biological effects. The following examples are intended to show the diversity of the presence of A₁ receptors rather than a comprehensive listing of all such receptors. Adenosine A₁ receptors are particularly ubiquitous within the central nervous system (CNS) with high levels being expressed in the cerebral cortex, hippocampus, cerebellum, thalamus, brain stem and spinal cord. Immuno-histochemical analysis using polyclonal antisera generated against rat and human adenosine A₁ receptors has identified different labeling densities of individual cells and their processes in selected regions of the brain. Adenosine A₁ receptor mRNA is widely distributed in peripheral tissues such as the vas deferens, testis, white adipose tissue, stomach, spleen, pituitary, adrenal, heart, aorta, liver, eye and bladder. Only very low levels of A₁ receptors are thought to be present in lung, kidney and small intestine.

Adenosine has been proposed for the treatment for pain states derived from nociception including acute pain, tissue injury pain and nerve injury pain. Adenosine modulates the pain response by stimulating adenosine A₁ receptors present in the dorsal root of the spinal cord and higher brain centers (spraspinal mechanisms). Adenosine A₁ agonists have been shown to be effective treatment for pain in animal pain models. However, A₁ agonists also cause cardiovascular side effects and CNS side effects such as heart block, hypotension and sedation.

More recently, the activation of adenosine A₁ receptors by an allosteric adenosine A₁ receptor enhancer, (2-amino-4,5,6,7-tetrahydrobenzo[b]thiophen-3-yl)(4-chlorophenyl)-methanone of the formula

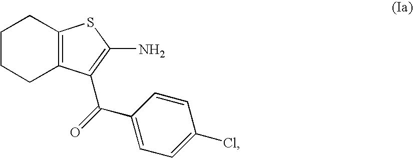

also known as T-62, has been demonstrated to reduce inflammatory and neuropathic pain and shown to be orally effective and devoid of the adverse side effects associated with administration of adenosine (Li et al., J. Pharmacol. Exp. Ther. 2003, 305, 950-955; U.S. Pat. No. 6,248,774 and No. 6,489,356)

### SUMMARY OF THE INVENTION

In one aspect, the present invention relates to pharmaceutical compositions, and oral dosage forms thereof, comprising an allosteric adenosine A₁ receptor enhancer and at least one pharmaceutically acceptable lipid excipient. More specifically, the present invention provides oral dosage forms comprising a 2-amino-3-aroylthiophene derivative, such as T-62, as the allosteric adenosine A₁ receptor enhancer, and at least one pharmaceutically acceptable lipid excipient, which dosage forms deliver the drug substance in a bioavailable manner.

In another aspect, the present invention relates to a method for the treatment of pain, including acute pain, e.g., postoperative pain, chronic pain, inflammatory pain, neuropathic pain and pain associated with migraine, in a subject, including man, in need thereof, which method comprises administering to the subject a pharmaceutical composition comprising a therapeutically effective amount of an allosteric adenosine A₁ receptor enhancer, e.g., a 2-amino-3-aroylthiophene derivative, such as T-62, or a pharmaceutically acceptable salt thereof, and at least one pharmaceutically acceptable lipid excipient.

Allosteric adenosine A₁ receptor enhancers, e.g., 2-amino-3-aroylthiophene derivatives, such as T-62, can be difficult to formulate due to their physico-chemical properties, such as low water solubility. Furthermore, 2-amino-3-aroylthiophene derived allosteric adenosine A₁ receptor enhancers are generally susceptible to degradation by acid, base, oxidation and light, and they are not always sufficiently stable during processing and storage, and have low oral bioavailability in traditional oral dosage forms, such as tablets. Thus, there is a need to develop stable pharmaceutical compositions, and oral dosage forms thereof, that deliver the drug substance to a subject, including man, such that the drug substance is absorbed by the subject at a therapeutically effective amount.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following. Abbreviations are those generally known in the art.

### BRIEF DESCRIPTION OF THE DRAWINGS

FIGS. 1 and 4 show arithmetic mean plasma T-62 concentration-time profiles after a single oral administration of escalating dose levels of T-62 to young healthy subjects (linear scale).

FIG. 2 shows arithmetic mean plasma T-62 concentration-time profiles by age after a single oral administration of 4×100 mg dosage of T-62 to young and elderly subjects (linear scale).

FIG. 3 shows arithmetic mean plasma T-62 concentration-time profiles by food intake after a single oral administration of 4×100 mg dosage of T-62 to young healthy subjects (linear scale).

FIG. 5 shows arithmetic mean plasma T-62 concentration-time profiles following repeated administration of 100 mg, 2×100 mg and 4×100 mg of T-62 every 12 hours through steady state conditions to adult healthy subjects (linear scale).

### DETAILED DESCRIPTION OF THE INVENTION

As described herein above, the present invention provides pharmaceutical compositions, and oral dosage forms thereof, comprising an allosteric adenosine A₁ receptor enhancer, e.g., a 2-amino-3-aroylthiophene derivative, such as T-62, processes for the manufacture of such pharmaceutical compositions and oral dosage forms, and their use as medicaments for the treatment of pain, including acute pain, e.g., postoperative pain, chronic pain, inflammatory pain, neuropathic pain and pain associated with migraine. More specifically, the present invention relates to pharmaceutical compositions comprising a 2-amino-3-aroylthiophene derivative, such as T-62, and at least one pharmaceutically acceptable lipid excipient.

Listed below are some of the definitions of various terms used herein to describe certain aspects of the present invention. However, the definitions used herein are those generally known in the art and apply to the terms as they are used throughout the specification unless they are otherwise limited in specific instances.

The term “allosteric adenosine A₁ receptor enhancer” as used herein refers to a class of compounds that appear to enhance adenosine A₁ receptor function by stabilizing the high affinity state of the receptor-G-protein complex. This property may be measured as an increase in radioligand binding to the adenosine A₁ receptor. An enhancer that increases agonist binding can do so by either accelerating the association of an agonist to the receptor, or by retarding the dissociation of the “receptor-ligand” complex and, therefore, must bind to a site different from the agonist recognition site. This putative site is termed as the allosteric site, and presumably, compounds that bind to this site and enhance the agonist effect are termed as “allosteric enhancers”.

The term “therapeutically effective amount” refers to an amount of a drug or a therapeutic agent that will elicit the desired biological or medical response of a tissue, system or an animal (including man) that is being sought by a researcher or clinician, e.g., provides significant analgesic activity. The “therapeutically effective amount” will vary depending on the compound, the disease and its severity and the age, weight, etc., of the subject to be treated.

The term “treatment” shall be understood as the management and care of a patient for the purpose of combating the disease, condition or disorder.

The term “pain-alleviating” shall be understood herein to include the expressions “pain-suppressing”, “pain-reducing” and “pain-inhibiting” as the present invention is applicable to the alleviation of existing pain as well as the suppression or inhibition of pain which would otherwise ensue from an imminent pain-causing event.

The term “subject” include, but are not limited to, humans, dogs, cats, horses, pigs, cows, monkeys, rabbits, mice and laboratory animals. The preferred subjects are humans.

The term “pharmaceutically acceptable salt” refers to a non-toxic salt commonly used in the pharmaceutical industry which may be prepared according to methods well-known in the art.

The term “lipid excipient” refers to a class of hydrocarbon-containing organic compounds which includes, but it is not limited to: fats; oils; waxes; sterols; mono-, di- and triglycerides; fatty acids; neutral fats; and compound lipids such as lipoproteins, glycolipids and phospholipids.

The term “alkyl” refers to a hydrocarbon chain having 1-20 carbon atoms, preferably 1-10 carbon atoms, and more preferably 1-7 carbon atoms. The hydrocarbon chain may be straight, as for a hexyl or n-butyl chain, or branched, as for example t-butyl, 2-methyl-pentyl, 3-propyl-heptyl. Exemplary alkyl groups include methyl, ethyl, propyl, isopropyl, n-butyl, t-butyl, isobutyl, pentyl, hexyl, isohexyl, heptyl, 4,4-dimethylpentyl, octyl, and the like.

The term “substituted alkyl” refers to those alkyl groups as described above substituted by one or more, preferably 1-3, of the following groups: halo, hydroxy, alkanoyl, alkoxy, cycloalkyl, cycloalkoxy, alkanoyloxy, thiol, alkylthio, alkylthiono, sulfonyl, sulfamoyl, carbamoyl, cyano, carboxy, acyl, aryl, aryloxy, alkenyl, alkynyl, aralkoxy, guanidino, optionally substituted amino, heterocyclyl including imidazolyl, furyl, thienyl, thiazolyl, pyrrolidyl, pyridyl, pyrimidyl and the like.

The term “lower alkyl” refers to those alkyl groups as described above having 1-6, preferably 1-4 carbon atoms.

The term “alkenyl” refers to any of the above alkyl groups having at least two carbon atoms and further containing a carbon-to-carbon double bond at the point of attachment. Groups having 2-6 carbon atoms are preferred.

The term “alkynyl” refers to any of the above alkyl groups having at least two carbon atoms and further containing a carbon-to-carbon triple bond at the point of attachment. Groups having 2-6 carbon atoms are preferred.

The term “alkylene” refers to a straight-chain bridge of 1-6 carbon atoms connected by single bonds, e.g., —(CH₂)_{X}—, wherein x is 1-6, in those cases where x is greater than 1, the chain may be interrupted with one or more groups selected from O, S, S(O), S(O)₂, CH═CH, C≡C or NR, wherein R may be hydrogen, alkyl, alkenyl, alkynyl, cycloalkyl, aryl, heterocyclyl, aralkyl, heteroaralkyl, acyl, carbamoyl, sulfonyl, alkoxycarbonyl, aryloxycarbonyl or aralkoxycarbonyl and the like; and the alkylene may further be substituted with one or more substituents selected from optionally substituted alkyl, cycloalkyl, aryl, heterocyclyl, oxo, halogen, hydroxy, carboxy, alkoxy, alkoxycarbonyl and the like.

The term “cycloalkyl” refers to monocyclic, bicyclic or tricyclic hydrocarbon groups of 3-12 carbon atoms, each of which may contain one or more carbon-to-carbon double bonds.

The term “substituted cycloalkyl” refers to those cycloalkyl groups as described above substituted by one or more substituents, preferably 1-3, such as alkyl, halo, oxo, hydroxy, alkoxy, alkanoyl, acylamino, carbamoyl, alkylamino, dialkylamino, thiol, alkylthio, cyano, carboxy, alkoxycarbonyl, sulfonyl, sulfonamido, sulfamoyl, heterocyclyl and the like.

Exemplary monocyclic hydrocarbon groups include, but are not limited to, cyclopropyl, cyclobutyl, cyclopentyl, cyclopentenyl, cyclohexyl, cyclohexenyl, 4,4-dimethylcyclohex-1-yl, cyclooctenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

In the definitions listed herein, when a reference to an alkyl, cycloalkyl, alkenyl or alkynyl group is made as part of the term, a substituted alkyl, cycloalkyl, alkenyl or alkynyl group is also intended.

The term “alkoxy” refers to alkyl-O—.

The term “cycloalkoxy” refers to cycloalkyl-O—.

The term “alkanoyl” refers to alkyl-C(O)—.

The term “cycloalkanoyl” refers to cycloalkyl-C(O)—.

The term “alkenoyl” refers to alkenyl-C(O)—.

The term “alkynoyl” refers to alkynyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carbamoyl and the like.

The term “aryl” refers to monocyclic or bicyclic aromatic hydrocarbon groups having 6-12 carbon atoms in the ring portion, such as phenyl, biphenyl, naphthyl, 2,3-dihydro-1H-indenyl and tetrahydronaphthyl.

The term “substituted aryl” refers to those aryl groups as described above substituted by 1-4 substituents in each ring portion, such as alkyl, trifluoromethyl, cycloalkyl, halo, hydroxy, alkoxy, methylenedioxy, acyl, alkanoyloxy, aryloxy, optionally substituted amino, thiol, alkylthio, arylthio, nitro, cyano, carboxy, alkoxycarbonyl, carbamoyl, alkylthiono, sulfonyl, sulfonamido, heterocyclyl and the like.

The term “monocyclic aryl” refers to optionally substituted phenyl as described above under aryl. Preferably, the monocyclic aryl is substituted by 1-3 substituents selected from the group consisting of halogen, cyano or trifluoromethyl.

In the definitions listed herein, when a reference to an aryl group is made as part of the term, a substituted aryl group is also intended.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl-S(O)₂—.

The term “arylthio” refers to aryl-S—.

The term “aroyl” refers to aryl-C(O)—.

The term “aroyloxy” refers to aryl-C(O)—O—.

The term “aroylamino” refers to aryl-C(O)—NH—.

The term “aryloxycarbonyl” refers to aryl-O—C(O)—.

The term “heterocyclyl” or “heterocyclo” refers to fully saturated or unsaturated, aromatic or nonaromatic cyclic group, e.g., which is a 4- to 7-membered monocyclic, 7- to 12-membered bicyclic or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl (pyridyl), pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “substituted heterocyclyl” refers to those heterocyclic groups described above substituted with 1, 2 or 3 substituents selected from the group consisting of the following:
(a) alkyl;
(b) hydroxyl (or protected hydroxyl);
(c) halo;
(d) oxo, i.e., ═O;
(e) optionally substituted amino;
(f) alkoxy;
(g) cycloalkyl;
(h) carboxy;
(i) heterocyclooxy;
(j) alkoxycarbonyl, such as unsubstituted lower alkoxycarbonyl;
(k) thiol;
(l) nitro;
(m) cyano;
(n) sulfamoyl;
(o) alkanoyloxy;
(p) aroyloxy;
(q) arylthio;
(r) aryloxy;
(s) alkylthio;
(t) formyl;
(u) carbamoyl;
(v) aralkyl; and
(w) aryl optionally substituted with alkyl, cycloalkyl, alkoxy, hydroxyl, amino, acylamino, alkylamino, dialkylamino or halo.

The term “heterocyclooxy” denotes a heterocyclic group bonded through an oxygen bridge.

The term “heterocycloalkyl” refers to nonaromatic heterocyclic groups as described above.

The term “heteroaryl” refers to an aromatic heterocycle, e.g., monocyclic or bicyclic aryl, such as pyrrolyl, pyrazolyl, imidazolyl, triazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl and the like, optionally substituted by, e.g., halogen, cyano, nitro, trifluoromethyl, lower alkyl or lower alkoxy.

The term “heterocycloalkanoyl” refers to heterocycloalkyl-C(O)—.

The term “heteroarylsulfonyl” refers to heteroaryl-S(O)₂—.

The term “heteroaroyl” refers to heteroaryl-C(O)—.

The term “heteroaroylamino” refers to heteroaryl-C(O)NH—.

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, cycloalkanoyl, alkenoyl, alkynoyl, aroyl, heterocycloalkanoyl, heteroaroyl, aralkanoyl, heteroaralkanoyl and the like.

The term “substituted acyl” refers to those acyl groups described above wherein the alkyl, cycloalkyl, alkenyl, alkynyl, aryl, heterocycloalkyl, heteroaryl, aralkyl or heteroaralkyl group is substituted as described herein above respectively.

The term “acylamino” refers to alkanoylamino, aroylamino, heteroaroylamino, aralkanoylamino, heteroaralkanoylamino and the like.

The term “halogen” or “halo” refers to fluorine, chlorine, bromine and iodine.

As noted herein above, allosteric adenosine A₁ receptor enhancers, e.g., 2-amino-3-aroylthiophene derivatives, such as T-62, can be difficult to formulate due to their physico-chemical properties, such as low water solubility. Furthermore, 2-amino-3-aroylthiophene derived allosteric adenosine A₁ receptor enhancers are generally susceptible to degradation by acid, base, oxidation and light, and they are not always sufficiently stable during processing and storage, or have low oral bioavailability in traditional oral dosage forms such as tablets. Thus, there is a need to develop stable pharmaceutical compositions, and oral dosage forms thereof, that deliver the drug substance to a subject, including man, such that the drug substance is absorbed by the subject at a therapeutically effective amount.

Suitable allosteric adenosine A₁ receptor enhancers to which the present invention applies include, but are not limited to, 2-amino-3-aroylthiophene derivatives, e.g., those disclosed in U.S. Pat. No. 6,323,214; No. 6,713,638; and No. 6,727,258; the entire contents of which are incorporated herein by reference.

Preferably, the allosteric adenosine A₁ receptor enhancer of the present invention is a 2-amino-3-aroylthiophene derivative selected from the group consisting of T-62 and, the compounds of formulae (Ib) and (Ic):

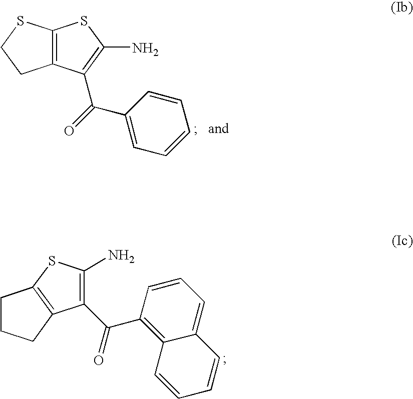

or in each case, a pharmaceutically acceptable salt thereof.

More preferably, the allosteric adenosine A₁ receptor enhancer of the present invention is T-62.

Suitable allosteric adenosine A₁ receptor enhancers also include 2-amino-3-aroylthiophene derivatives of the formula

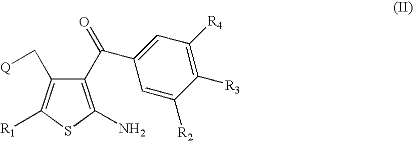

wherein
R₁ is hydrogen, alkyl, substituted alkyl, aryl, substituted aryl, cycloalkyl or substituted cycloalkyl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy or substituted alkoxy;
Q is selected from the group consisting of

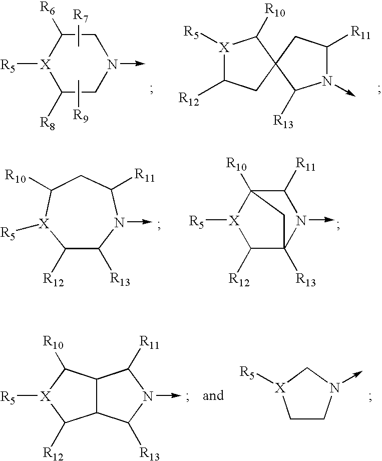

in which
   R₅ is hydrogen, alkyl, substituted alkyl, aryl, substituted aryl, cycloalkyl, substituted cycloalkyl, heteroaryl, substituted heteroaryl, heteroaralkyl, substituted heteroaralkyl, acyl or substituted acyl;
   R₆ and R₇ are, independently from each other, hydrogen, C₁-C₃ alkyl or C₁-C₃ substituted alkyl; or
   R₆ and R₇, provided they are attached to the same carbon atom, combined are alkylene which together with the carbon atom to which they are attached form a 3- to 7-membered spirocyclic ring;
   R₈, R₉, R₁₀, R₁₁, R₁₂ and R₁₃ are, independently from each other, hydrogen, C₁-C₃ alkyl or C₁-C₃ substituted alkyl;
   X is N or C—H; or
   X is C—NR₁₄R₁₅ wherein R₁₄ and R₁₅ are, independently from each other, hydrogen, C₁-C₃ alkyl, C₁-C₃ substituted alkyl, aryl or substituted aryl; or
   X is C—R₁₆ wherein R₁₆ and R₅ combined are a carbonyl oxygen; or
   X is C—R₁₆ wherein R₁₆ and R₅ combined are a divalent radical of the formula

←Y—CHR₁₇—(CH₂)ₙ—CHR₁₈—Y→
which together with the carbon atom to which R₁₆ and R₅ are attached form a 5- to 7-membered spirocyclic ring, and in which
   Y is oxygen or sulfur;
   R₁₇ and R₁₈ are, independently from each other, hydrogen, C₁-C₆ alkyl, C₁-C₆ substituted alkyl, cycloalkyl, substituted cycloalkyl, aryl or substituted aryl;
   n is zero, or an integer of 1 or 2; or
X is C—R₁₆ wherein R₁₆ and R₅ combined are a divalent radical of the formula

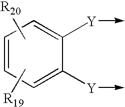

which together with the carbon atom to which R₁₆ and R₅ are attached form a 5-membered spirocyclic ring, and in which
   Y is oxygen or sulfur;
   R₁₉ and R₂₀ are, independently from each other, hydrogen, halogen,
   cyano, trifluoromethyl, C₁-C₆ alkyl, C₁-C₆ substituted alkyl or C₁-C₆ alkoxy;
      or a pharmaceutically acceptable salt thereof.

Preferred are the compounds of formula (II), wherein
R₁ is hydrogen, alkyl, substituted alkyl, aryl or substituted aryl; or a pharmaceutical composition thereof.

Further preferred are the compounds of formula (II), designated as the A group, wherein Q is

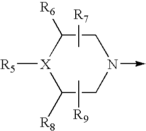

in which
R₅ is hydrogen, alkyl, substituted alkyl, aryl, substituted aryl, cycloalkyl, substituted cycloalkyl, heteroaryl, substituted heteroaryl, heteroaralkyl, substituted heteroaralkyl, acyl, or substituted acyl;
R₆ and R₇ are, independently from each other, hydrogen, C₁-C₃ alkyl, or C₁-C₃ substituted alkyl; or
R₆ and R₇, provided they are attached to the same carbon atom, combined are alkylene which together with the carbon atom to which they are attached form a 3- to 7-membered spirocyclic ring;
X is N or C—H; or
X is C—NR₁₄R₁₅ wherein R₁₄ and R₁₅ are, independently from each other, hydrogen, C₁-C₃ alkyl, C₁-C₃ substituted alkyl, aryl, or substituted aryl; or
X is C—R₁₆ wherein R₁ and R₅ combined are a carbonyl oxygen; or
X is C—R₁₆ wherein R₁₆ and R₅ combined are a divalent radical of the formula

←Y—CHR₁₇—(CH₂)ₙ—CHR₁₈—Y→
which together with the carbon atom to which R₁₆ and R₅ are attached form a 5- to 7-membered spirocyclic ring, and in which
   Y is oxygen or sulfur;
   R₁₇ and R₁₈ are, independently from each other, hydrogen, C₁-C₆ alkyl, C₁-C₆ substituted alkyl, cycloalkyl, substituted cycloalkyl, aryl, or substituted aryl;
   n is zero, or an integer of 1 or 2; or
X is C—R₁₆ wherein R₁₆ and R₅ combined are a divalent radical of the formula

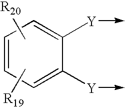

which together with the carbon atom to which R₁₆ and R₅ are attached form a 5-membered spirocyclic ring, and in which
   Y is oxygen or sulfur;
   R₁₉ and R₂₀ are, independently from each other, hydrogen, halogen, cyano, trifluoromethyl, C₁-C₆ alkyl, C₁-C₆ substituted alkyl, or C₁-C₆ alkoxy;
      or a pharmaceutically acceptable salt thereof.

Preferred are the compounds in the A group, designated as the B group, wherein
X is N;
   or a pharmaceutically acceptable salt thereof.

Preferred are the compounds in the B group having formula (IIA)

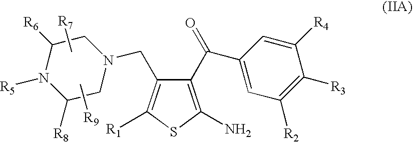

wherein
R₁ is hydrogen, alkyl, substituted alkyl, aryl or substituted aryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy;
R₅ is alkyl, substituted alkyl, aryl, substituted aryl, cycloalkyl, substituted cycloalkyl, heteroaryl, substituted heteroaryl, heteroaralkyl, substituted heteroaralkyl, acyl, or substituted acyl;
R₆ and R₇ are, independently from each other, hydrogen, C₁-C₃ alkyl, or C₁-C₃ substituted alkyl; or
R₆, and R₇, provided they are attached to the same carbon atom, combined are alkylene which together with the carbon atom to which they are attached form a 3- to 7-membered spirocyclic ring;
R₈ and R₉ are, independently from each other, hydrogen, C₁-C₃ alkyl, or C₁-C₃ substituted alkyl;
   or a pharmaceutically acceptable salt thereof.

Preferred are the compounds of formula (IIA) wherein
R₁ is hydrogen or C₁-C₃ alkyl;
   or a pharmaceutically acceptable salt thereof.

Preferred are also the compounds of formula (IIA) wherein
R₅ is monocyclic aryl optionally substituted by one to three substituents selected from the group consisting of halogen, cyano or trifluoromethyl;
   or a pharmaceutically acceptable salt thereof.

Preferred are also the compounds of formula (IIA), designated as the C group, wherein
R₂ and R₄ are hydrogen;
   or a pharmaceutically acceptable salt thereof.

Preferred are the compounds in the C group wherein
R₃ is halogen, cyano or trifluoromethyl;
   or a pharmaceutically acceptable salt thereof.

Preferred are also the compounds of formula (IIA), designated as the D group, wherein
R₂ and R₃ are hydrogen;
   or a pharmaceutically acceptable salt thereof.

Preferred are the compounds in the D group wherein
R₄ is halogen, cyano or trifluoromethyl;
   or a pharmaceutically acceptable salt thereof.

Preferred are also the compounds of formula (IIA), designated as the E group, wherein
R₆, R₇, R₈ and R₉ are, independently from each other, hydrogen, or C₁-C₃ alkyl;
   or a pharmaceutically acceptable salt thereof.

Preferred are the compounds in the E group, designated as the F group, wherein
R₅ is monocyclic aryl optionally substituted by one to three substituents selected from the group consisting of halogen, cyano, or trifluoromethyl;
   or a pharmaceutically acceptable salt thereof.

Preferred are the compounds in the F group, designated as the G group, wherein
R₂ and R₄ are hydrogen;
   or a pharmaceutically acceptable salt thereof.

Preferred are the compounds in the G group wherein
R₃ is halogen, cyano or trifluoromethyl;
   or a pharmaceutically acceptable salt thereof.

Further preferred are the compounds in the G group wherein
R₁ is hydrogen or C₁-C₃ alkyl;
   or a pharmaceutically acceptable salt thereof.

Preferred are also the compounds in the F group, designated as the H group, wherein
R₂ and R₃ are hydrogen;
   or a pharmaceutically acceptable salt thereof.

Preferred are the compounds in the H group wherein
R₄ is halogen, cyano or trifluoromethyl;
   or a pharmaceutically acceptable salt thereof.

Further preferred are the compounds in the H group wherein
R₁ is hydrogen or C₁-C₃ alkyl;
   or a pharmaceutically acceptable salt thereof.

Preferred are also the compounds in the A group, designated as the I group, wherein
X is C—R₁₆ wherein R₁₆ and R₅ combined are a divalent radical of the formula

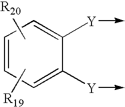

which together with the carbon atom to which R₁₆ and R₅ are attached form a 5-membered spirocyclic ring, and in which
   Y is oxygen;
   R₁₉ and R₂₀ are, independently from each other, hydrogen, halogen, cyano, trifluoromethyl, C₁-C₆ alkyl, C₁-C₆ substituted alkyl, or C₁-C₆ alkoxy;
      or a pharmaceutically acceptable salt thereof.

Preferred are the compounds in the I group having formula (IIB)

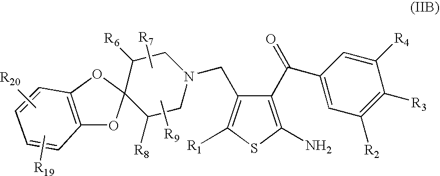

wherein
R₁ is hydrogen, alkyl, substituted alkyl, aryl or substituted aryl;
R₂, R₃, and R₄ are, independently from each other, hydrogen, alkyl, substituted alkyl, aryl, substituted aryl, cycloalkyl, substituted cycloalkyl, halogen, hydroxyl, nitro, cyano, alkoxy, or substituted alkoxy;
R₆, R₇, R₈ and R₉ are, independently from each other, hydrogen, C₁-C₃ alkyl, or C₁-C₃ substituted alkyl; or
R₁₉ and R₂₀ are, independently from each other, hydrogen, halogen, cyano, trifluoromethyl, C₁-C₆ alkyl, C₁-C₆ substituted alkyl, or C₁-C₆ alkoxy;
   or a pharmaceutically acceptable salt thereof.

Preferred are the compounds of formula (IIB) wherein
R₁ is hydrogen or C₁-C₃ alkyl;
   or a pharmaceutically acceptable salt thereof.

Preferred are also the compounds of formula (IIB) wherein
R₁₉ and R₂₀ are, independently from each other, hydrogen, halogen, cyano, trifluoromethyl, or C₁-C₄ alkyl;
   or a pharmaceutically acceptable salt thereof.

Preferred are also the compounds of formula (IIB), designated as the J group, wherein
R₂ and R₄ are hydrogen;
   or a pharmaceutically acceptable salt thereof.

Preferred are the compounds in the J group wherein
R₃ is halogen, cyano or trifluoromethyl;
   or a pharmaceutically acceptable salt thereof.

Preferred are also the compounds of formula (IIB), designated as the K group, wherein
R₂ and R₃ are hydrogen;
   or a pharmaceutically acceptable salt thereof.

Preferred are the compounds in the K group wherein
R₄ is halogen, cyano or trifluoromethyl;
   or a pharmaceutically acceptable salt thereof.

Preferred are also the compounds of formula (IIB), designated as the L group, wherein
R₆, R₇, R₈ and R₉ are hydrogen;
   or a pharmaceutically acceptable salt thereof.

Preferred are the compounds in the L group, designated as the M group, wherein
R₁₉ and R₂₀ are, independently from each other, hydrogen, halogen, cyano, trifluoromethyl, or C₁-C₄ alkyl;
   or a pharmaceutically acceptable salt thereof.

Preferred are the compounds in the M group, designated as the N group, wherein
R₂ and R₄ are hydrogen;
   or a pharmaceutically acceptable salt thereof.

Preferred are the compounds in the N group wherein
R₃ is halogen, cyano or trifluoromethyl;
   or a pharmaceutically acceptable salt thereof.

Further preferred are the compounds in the N group wherein
R₁ is hydrogen or C₁-C₃ alkyl;
   or a pharmaceutically acceptable salt thereof.

Preferred are also the compounds in the M group, designated as the O group, wherein
R₂ and R₃ are hydrogen;
   or a pharmaceutically acceptable salt thereof.

Preferred are the compounds in the O group wherein
R₄ is halogen, cyano or trifluoromethyl;
   or a pharmaceutically acceptable salt thereof.

Further preferred are the compounds in the O group wherein
R₁ is hydrogen or C₁-C₃ alkyl;
   or a pharmaceutically acceptable salt thereof.

Specific examples of allosteric adenosine A₁ receptor enhancers of formula (II) include:
{2-Amino-4-[(4-phenylpiperazin-1-yl)methyl]thiophen-3-yl}(4-chlorophenyl)methanone;
{2-Amino-4-[(4-methylpiperazin-1-yl)methyl]thiophen-3-yl}(4-chlorophenyl)methanone;
{2-Amino-4-[4-((4-fluorophenyl)piperazin-1-yl)methyl]thiophen-3-yl}(4-chlorophenyl)methanone;
{2-Amino-4-[4-((4-chlorophenyl)piperazin-1-yl)methyl]thiophen-3-yl}(4-chlorophenyl)methanone;
{2-Amino-4-[4-((4-methoxyphenyl)piperazin-1-yl)methyl]thiophen-3-yl}(4-chlorophenyl)methanone;
{2-Amino-4-[(4-p-tolylpiperazin-1-yl)methyl]thiophen-3-yl}(4-chlorophenyl)methanone;
{2-Amino-4-[(4-(pyridin-2-yl)piperazin-1-yl)methyl]thiophen-3-yl}(4-chlorophenyl)methanone;
{2-Amino-4-[(4-(pyrimidin-2-yl)piperazin-1-yl)methyl]thiophen-3-yl}(4-chlorophenyl)methanone;
{2-Amino-4-[(4-(3,4-dichlorophenyl)-piperazin-1-yl)methyl]thiophen-3-yl}(4-chlorophenyl)methanone;
4-{4-[(5-Amino-4-(4-chlorobenzoyl)thiophen-3-yl)methyl]piperazin-1-yl}benzonitrile;
{2-Amino-4-[(4-(3-chlorophenyl)-piperazin-1-yl)methyl]thiophen-3-yl}(4-chlorophenyl)methanone;
{2-Amino-4-[(4-(2-chlorophenyl)piperazin-1-yl)methyl]thiophen-3-yl}(4-chlorophenyl)methanone;
{2-Amino-4-[(4-(2-fluorophenyl)-piperazin-1-yl)methyl]thiophen-3-yl}(4-chlorophenyl)methanone;
{2-Amino-4-[(4-(3-(trifluoromethyl)phenyl)piperazin-1-yl)methyl]thiophen-3-yl}(4-chlorophenyl)methanone;
1-{4-[(5-Amino-4-(4-chlorobenzoyl)thiophen-3-yl)methyl]piperazin-1-yl}-2-(4-chlorophenyl)ethanone;
{2-Amino-4-[(4-(4-chlorobenzoyl)piperazin-1-yl)methyl]thiophen-3-yl}(4-chlorophenyl)methanone;
{2-Amino-4-[(4-(pyridin-4-yl)piperazin-1-yl)methyl]thiophen-3-yl}(4-chlorophenyl)methanone;
{2-Amino-4-[(4-(benzo[d][1,3]dioxol-5-yl)piperazin-1-yl)methyl]thiophen-3-yl}(4-chlorophenyl)methanone;
{2-Amino-4-[(4-(2,3-dichlorophenyl)piperazin-1-yl)methyl]thiophen-3-yl}(4-chlorophenyl)methanone;
{2-Amino-4-[(4-(3-fluorophenyl)piperazin-1-yl)methyl]thiophen-3-yl}(4-chlorophenyl)methanone;
{2-Amino-4-[(4-(3,5-dichlorophenyl)piperazin-1-yl)methyl]thiophen-3-yl}(4-chlorophenyl)methanone;
{2-Amino-4-[(4-(4-(trifluoromethyl)phenyl)piperazin-1-yl)methyl]thiophen-3-yl}(4-chlorophenyl)methanone;
2-{4-[(5-Amino-4-(4-chlorobenzoyl)thiophen-3-yl)methyl]piperazin-1-yl}-1-(4-chlorophenyl)ethanone;
{2-Amino-4-[(4-(2,4-difluorophenyl)piperazin-1-yl)methyl]thiophen-3-yl}(4-chlorophenyl)methanone;
{2-Amino-4-[(4-(2,6-difluorophenyl)piperazin-1-yl)methyl]thiophen-3-yl}(4-chlorophenyl)methanone;
{2-Amino-4-[(4-(3-chloro-4-fluorophenyl)piperazin-1-yl)methyl]thiophen-3-yl}(4-chlorophenyl)methanone;
{2-Amino-4-[(4-cyclohexylpiperazin-1-yl)methyl]thiophen-3-yl}(4-chlorophenyl)methanone;
{2-Amino-4-[(4-(4-chlorophenyl)piperidin-1-yl)methyl]thiophen-3-yl}(4-chlorophenyl)methanone;
{2-Amino-4-[(4-(4-nitrophenyl)piperazin-1-yl)methyl]thiophen-3-yl}(4-chlorophenyl)methanone;
{2-Amino-4-[(4-isopropyl)piperazin-1-yl)methyl]thiophen-3-yl}(4-chlorophenyl)methanone;
{2-Amino-4-[(4-naphthalen-1-yl)piperazin-1-yl)methyl]thiophen-3-yl}(4-chlorophenyl)methanone;
{2-Amino-4-[(4-(3,4-difluorophenyl)piperazin-1-yl)methyl]thiophen-3-yl}(4-chlorophenyl)methanone;
{2-Amino-4-[(4-cyclopentylpiperazin-1-yl)methyl]thiophen-3-yl}(4-chlorophenyl)methanone;
{2-Amino-4-[(4-cycloheptylpiperazin-1-yl)methyl]thiophen-3-yl}(4-chlorophenyl)methanone;
{2-Amino-4-[(4-(4-chlorobenzyl)piperazin-1-yl)methyl]thiophen-3-yl}(4-chlorophenyl)methanone;
{2-Amino-4-[(4-benzylpiperazin-1-yl)methyl]thiophen-3-yl}(4-chlorophenyl)methanone;
(2-Amino-4-{[4-(2-(4-chlorophenyl)ethyl)piperazin-1-yl]methyl}thiophen-3-yl)(4-chlorophenyl)methanone;
{2-Amino-4-[(4-(4-fluorobenzyl)piperazin-1-yl)methyl]thiophen-3-yl}(4-chlorophenyl)methanone;
{2-Amino-4-[(4-cyclooctylpiperazin-1-yl)methyl]thiophen-3-yl}(4-chlorophenyl)methanone;
(2-Amino-4-{[4-[3-(4-chlorophenyl)propyl]piperazin-1-yl]methyl}thiophen-3-yl)(4-chlorophenyl)methanone;
{2-Amino-4-[(4-(2,4-dichlorophenyl)piperazin-1-yl)methyl]thiophen-3-yl}(4-chlorophenyl)methanone;
{2-Amino-4-[(4-(2,5-difluorophenyl)piperazin-1-yl)methyl]thiophen-3-yl}(4-chlorophenyl)methanone;
{2-Amino-4-[(4-(2-(trifluoromethyl)phenyl)piperazin-1-yl)methyl]thiophen-3-yl}(4-chlorophenyl)methanone;
{2-Amino-4-[(4-(4-chloro-3-(trifluoromethyl)phenyl)piperazin-1-yl)methyl]thiophen-3-yl}(4-chlorophenyl)methanone;
{2-Amino-4-[(4-(2,4,6-trifluorophenyl)piperazin-1-yl)methyl]thiophen-3-yl}(4-chlorophenyl)methanone;
{2-Amino-4-[(4-(2-chloro-4-fluorophenyl)piperazin-1-yl)methyl]thiophen-3-yl}(4-chlorophenyl)methanone;
{2-Amino-4-[(4-(2-fluoro-4-chlorophenyl)piperazin-1-yl)methyl]thiophen-3-yl}(4-chlorophenyl)methanone;
{2-Amino-4-[(4-(3,5-difluorophenyl)piperazin-1-yl)methyl]thiophen-3-yl}(4-chlorophenyl)methanone;
{2-Amino-4-[(4-(2,6-dichlorophenyl)piperazin-1-yl)methyl]thiophen-3-yl}(4-chlorophenyl)methanone;
{2-Amino-4-[(4-(4-(trifluoromethoxy)phenyl)piperazin-1-yl)methyl]thiophen-3-yl}(4-chlorophenyl)methanone;
{2-Amino-4-[(4-(pyridin-3-yl)piperazin-1-yl)methyl]thiophen-3-yl}(4-chlorophenyl)methanone;
{2-Amino-4-[(4-(2,5-dichlorophenyl)piperazin-1-yl)methyl]thiophen-3-yl}(4-chlorophenyl)methanone;
{2-Amino-4-[(4-(2,3-difluorophenyl)piperazin-1-yl)methyl]thiophen-3-yl}(4-chlorophenyl)methanone;
{2-Amino-4-[(4-(4-chlorophenyl)-3-methylpiperazin-1-yl)methyl]thiophen-3-yl}(4-chlorophenyl)methanone;
{2-Amino-4-[(4-(4-(trifluoromethyl)phenyl)piperazin-1-yl)methyl]thiophen-3-yl}[3-(trifluoromethyl)phenyl]methanone;
{2-Amino-4-[(4-(3-fluorophenyl)piperazin-1-yl)methyl]thiophen-3-yl}[3-(trifluoromethyl)phenyl]methanone;
{2-Amino-4-[(4-(2,6-difluorophenyl)piperazin-1-yl)methyl]thiophen-3-yl}[3-(trifluoromethyl)phenyl]methanone;
{2-Amino-4-(spiro[benzo[d][1,3]-dioxole-2,4′piperidine]-1′-ylmethyl)thiophen-3-yl}(4-chlorophenyl)methanone;
{2-Amino-4-(5-tert-butylspiro[benzo[d][1,3]-dioxole-2,4′-piperidine]-1′-ylmethyl)thiophen-3-yl}(4-chlorophenyl)methanone;
{2-Amino-4-(4-fluorospiro[benzo[d][1,3]-dioxole-2,4′-piperidine]-1′-ylmethyl)thiophen-3-yl}(4-chlorophenyl)methanone;
{2-Amino-4-(4-methylspiro[benzo[d][1,3]-dioxole-2,4′piperidine]-1′-ylmethyl)thiophen-3-yl}(4-chlorophenyl)methanone;
{2-Amino-4-(5-methylspiro[benzo[d][1,3]-dioxole-2,4′-piperidine]-1′-ylmethyl)thiophen-3-yl}(4-chlorophenyl)methanone;
{2-Amino-4-[4-((4-chlorophenylamino)piperidin-1-yl)methyl]thiophen-3-yl}(4-chlorophenyl)methanone;
{2-Amino-4-[(4-(4-chlorophenyl)methylamino]piperidin-1-yl)methyl]thiophen-3-yl}(4-chlorophenyl)methanone;
{2-Amino-4-[(4-(4-chlorophenyl)-[1,4]diazepan-1-yl)methyl]thiophen-3-yl}(4-chlorophenyl)methanone;
{2-Amino-4-[(7-(4-chlorophenyl)-2,7-diaza-spiro[4.4]non-2-yl)methyl]thiophen-3-yl}(4-chlorophenyl)methanone;
{2-Amino-4-[(5-(4-chlorophenyl)hexahydropyrrolo[3,4-c]pyrrol-2-yl)methyl]thiophen-3-yl}(4-chlorophenyl)methanone;
{2-Amino-4-[(5-(4-chlorophenyl)-2,5-diazabicyclo[2.2.1]hept-2-yl)methyl]thiophen-3-yl}(4-chlorophenyl)methanone;
{2-Amino-4-[(4-(4-fluorophenyl)piperazin-1-yl)methyl]-5-methylthiophen-3-yl}(4-chlorophenyl)methanone;
{2-Amino-5-methyl-4-[(4-phenylpiperazin-1-yl)methyl]thiophen-3-yl}(4-chlorophenyl)methanone;
{2-Amino-5-methyl-4-[(4-(4-(trifluoromethyl)phenyl)piperazin-1-yl)methyl]thiophen-3-yl}(4-chlorophenyl)methanone;
{2-Amino-4-[(4-(4-chlorophenyl)piperazin-1-yl)methyl]-5-methylthiophen-3-yl}(4-chlorophenyl)methanone;
{2-Amino-4-[(4-(4-bromophenyl)piperazin-1-yl)methyl]-5-methylthiophen-3-yl}(4-chlorophenyl)methanone;
{2-Amino-4-[(4-(4-iodophenyl)piperazin-1-yl)methyl]-5-methylthiophen-3-yl}(4-chlorophenyl)methanone;
{2-Amino-5-methyl-4-[(4-(4-nitrophenyl)piperazin-1-yl)methyl]thiophen-3-yl}(4-chlorophenyl)methanone;
4-{4-[(5-Amino-4-(4-chlorobenzoyl)-2-methylthiophen-3-yl)methyl]piperazin-1-yl}benzonitrile
{2-Amino-4-[(4-benzylpiperazin-1-yl)methyl)-5-methylthiophen-3-yl](4-chlorophenyl)methanone;
{2-Amino-4-[(4-(4-methoxyphenyl)piperazin-1-yl)methyl]-5-methylthiophen-3-yl}(4-chlorophenyl)methanone;
{2-Amino-5-methyl-4-[(4-p-tolylpiperazin-1-yl)methyl]thiophen-3-yl}(4-chlorophenyl)methanone;
{2-Amino-4-[(4-(3,4-dichlorophenyl)piperazin-1-yl)methyl]-5-methylthiophen-3-yl}(4-chlorophenyl)methanone;
{2-Amino-5-methyl-4-[(4-(3-(trifluoromethyl)phenyl)piperazin-1-yl)methyl]thiophen-3-yl}(4-chlorophenyl)methanone;
{2-Amino-4-[(4-(3-chlorophenyl)piperazin-1-yl)methyl]-5-methylthiophen-3-yl}(4-chlorophenyl)methanone;
{2-Amino-4-[(4-(4-chloro-3-(trifluoromethyl)phenyl)piperazin-1-yl)methyl]-5-methylthiophen-3-yl}(4-chlorophenyl)methanone;
{2-Amino-5-phenyl-4-[(piperidin-1-yl)methyl]thiophen-3-yl}(4-chlorophenyl)methanone;
{2-Amino-4-[4-(4-fluorophenyl)piperazin-1-yl)methyl]-5-ethylthiophen-3-yl}(4-chlorophenyl)methanone;
{2-Amino-5-ethyl-4-[(4-phenylpiperazin-1-yl)methyl]thiophen-3-yl}(4-chlorophenyl)methanone;
{2-Amino-4-[(4-(4-chlorophenyl)piperazin-1-yl)methyl]-5-ethylthiophen-3-yl}(4-chlorophenyl)methanone; and
{2-Amino-4-[(4-(3-fluorophenyl)piperazin-1-yl)methyl]-5-methylthiophen-3-yl}(4-chlorophenyl)methanone;
   or a pharmaceutically acceptable salt thereof.

The allosteric adenosine A₁ receptor enhancers, e.g., 2-amino-3-aroylthiophene derivatives, such as T-62, may be prepared using methods well known in the art, e.g., T-62, and the compounds of formulae (Ib) and (Ic) may be prepared using methods disclosed in U.S. Pat. No. 6,323,214; No. 6,713,638; and No. 6,727,258; or as described by Corral et al. in Afinidad 1978, 35(354), 129-33. Compounds of formulae (II), (IIA) and (IIB) may prepared, e.g., using methods disclosed in U.S. Patent Application Publication No. 20080119460.

As indicated herein above, the allosteric adenosine A₁ receptor enhancers may be present as their pharmaceutically acceptable salts. As well known in the art, a compound having at least one basic center such as an amino group, may form acid addition salts thereof. Similarly, a compound having at least one acidic group (for example —COOH) may form salts with bases.

In view of the foregoing, a person skilled in the art is fully enabled to identify, manufacture, and test allosteric adenosine A₁ receptor enhancers, or their pharmaceutically acceptable salts thereof, for their properties and efficacy in standard test models well known in the art, both in vitro and in vivo. For example, in vivo drug efficacy may be assessed using pain models such as carrageenan model (Guilbaud and Kayser, Pain 1987, 28, 99-107) for acute inflammatory pain, FCA model (Freund's Complete Adjuvant; Hay et al., Neuroscience 1997, 78(3), 843-850) for chronic inflammatory pain, CCl model (Chronic Constriction Injury; Bennett and Xie, Pain 1988, 33, 87-107) for neuropathic pain, or postincisional hypersensitivity model (Obata et al., Anesthesiology 2004, 100, 1258-1262) for postoperative pain.

As described herein above, the present invention provides pharmaceutical compositions, and oral dosage forms thereof, comprising an allosteric adenosine A₁ receptor enhancer, e.g., a 2-amino-3-aroylthiophene derivative, such as T-62, or a pharmaceutically acceptable salt thereof, and at least one pharmaceutically acceptable lipid excipient. Said compositions may contain from about 0.1% to about 90%, preferably from about 1% to about 80%, more preferably from about 1% to about 10%, and most preferably from about 4% to about 9% of the drug substance based on the total weight of the pharmaceutical composition. The pharmaceutical compositions of the present invention may take the form of solutions, suspensions, microemulsions, and the like. Preferably, the pharmaceutical compositions of the present invention are solutions. More preferably, the pharmaceutical compositions of the present invention are solutions that self-microemulsify upon dilution with aqueous media, e.g., under the gentle digestive motility of the stomach and the gastrointestinal (GI) tract.

Examples of pharmaceutically acceptable lipids include fats; oils; waxes; sterols; mono-, di- and triglycerides; fatty acids; neutral fats; and compound lipids such as lipoproteins, glycolipids and phospholipids. Additional non-limiting examples include glyceryl stearates (available from Sasol under the tradename IMWITOR®), polyoxyethylated oleic glycerides (available from Gattefosse, S.A., Saint Priest, France, under the tradename LABRAFIL®), mineral oil, and dimethylpolysiloxanes such as simethicone. Preferred pharmaceutical compositions of the present invention include the use of one or more oils, including vegetable oils such as soybean, corn and canola oil, more preferably, super refined soybean oil (USP). Preferably, the lipid excipient(s) is present in an amount of more than about 5% by weight based on the total weight of the pharmaceutical composition. Specific compositions of the present invention may contain about 5%, about 10%, about 12%, about 15%, about 20%, about 25%, about 30%, about 35%, about 40%, about 45%, about 50%, about 55%, about 60%, about 65%, about 70%, about 75%, about 80%, about 85%, about 90%, about 95% or about 98% of at least one pharmaceutically acceptable lipid excipient, based on the total weight of the pharmaceutical composition. Preferred embodiments include pharmaceutical compositions comprising from about 10% to about 30% of at least one pharmaceutically acceptable lipid excipient, more preferably, from about 12% to about 25% of at least one pharmaceutically acceptable lipid excipient, based on the total weight of the pharmaceutical composition.

Additionally, other excipients may be added to the compositions of the present invention. Such excipients include, but are not limited to, emulsifiers and excipients that solubilize the drug substance. Surfactants are frequently employed emulsifiers, and solubilizing agents include, but are not limited to, solvents.

Examples of surfactants include, but are not limited to, sodium lauryl sulfate, stearic acid, oleic acid, monoethanolamine, docusate sodium, sorbitan fatty acid esters, polyoxyethylene sorbitan fatty acid esters, ethoxylated aliphatic alcohols, propylene glycol monocaprylate (available, e.g., from Gattefosse Canada Inc. under the trade name CAPRYOL 90®), propylene glycol monolaurate (available from Abitec Corp., Columbus, Ohio, under the tradename CAPMUL®), glycerol monostearate, medium chain triglycerides, polyoxyethylene alkyl ethers, polysorbates (available, e.g., from ICI under the trade name TWEEN®), preferably polysorbate 80 (available, e.g., from Croda Inc. under the trade name CRILLET 4HP®), sorbitan monoesters (available, e.g., from ICI under the trade name SPAN®), caprylocaproyl macrogol-8 (available, e.g., from Gattefosse S.A., Saint Priest, France under the trade name LABRASOL®), cremophores, polyoxyethylene stearates, glyceryl monooleate, glyceryl monocaprate, glyceryl monocaprylate, glyceryl monostearate and mixtures thereof. These surfactants may be used alone, or in combinations thereof, in the pharmaceutical compositions of the present invention. It is contemplated that mixtures of hydrophilic and lipophilic surfactants may be used in the pharmaceutical compositions of the present invention. It is contemplated that the pharmaceutical compositions of the present invention may form microemulsions when the drug substance is combined with the lipid excipient and the one or more surfactants. The pharmaceutical compositions of the present invention may contain surfactant(s) in a total amount of about 1% to about 90% based on the total weight of the pharmaceutical composition. Specific embodiments of the present invention may contain about 1%, about 5%, about 10%, about 15%, about 20%, about 25%, about 30%, about 35%, about 40%, about 45%, about 50%, about 55%, about 60%, about 65%, about 70%, about 75%, about 80%, about 85% or about 90% of surfactant(s), based on the total weight of the pharmaceutical composition. Preferred embodiments may contain from about 10% to about 90% of surfactant(s), more preferably from about 65% to about 85% of surfactant(s), based on the total weight of the pharmaceutical composition. Preferred surfactants include caprylocaproyl macrogol-8, polysorbate 80 and propylene glycol monocaprylate, and mixtures thereof.

Examples of solvents include ethanol, benzyl alcohol, benzyl benzoate, ethyl acetate, ethyl oleate, glycofurol, isopropyl myristate and isopropyl palmitate.

The compositions of the present invention may include other standard pharmaceutical excipients, including plasticizers, crystallization inhibitors, wetting agents, bulk filling agents, bioavailability enhancers, pH-adjusting agents and combinations thereof. In addition, the compositions may be sterilized and/or contain preserving and stabilizing agents, or solution promoters, salts for regulating the osmotic pressure and/or buffers. Furthermore, they may also contain other therapeutically valuable substances.

A preferred pharmaceutical composition of the present invention includes a 2-amino-3-aroylthiophene derivative, such as T-62, mixed with super refined soybean oil (USP), propylene glycol monocaprylate (CAPRYOL 90®), caprylocaproyl macrogol-8 glycerides (LABRASOL®) and polysorbate 80 (CRILLET 4 HP®), and may optionally comprise ethanol. For example, a preferred composition of the present invention may comprise from about 4% to about 9% of the allosteric adenosine A₁ receptor enhancer T-62; from about 12% to about 25% of super refined soybean oil (USP); from about 41% to about 46% of propylene glycol monocaprylate (CAPRYOL 90®); from about 16% to about 30% of caprylocaproyl macrogol-8 glycerides (LABRASOL®); and from about 8% to about 9% of polysorbate 80 (CRILLET 4 HP®); based on the total weight of the pharmaceutical composition.

The pharmaceutical compositions of the present invention comprising an allosteric adenosine A₁ receptor enhancer, e.g., a 2-amino-3-aroylthiophene derivative, such as T-62, may be manufactured using conventional formulating methods known in the art. Preferably, the allosteric adenosine A₁ receptor enhancer, e.g., a 2-amino-3-aroylthiophene derivative, such as T-62, is first milled and then added to a mixture of propylene glycol monocaprylate (CAPROYL 90®), caprylocaproyl macrogol-8 glycerides (LABRASOL®), and polysorbate 80 (CRILLET 4 HP®) at 45° C.±5° C. while mixing and sparging with nitrogen throughout the process. Super refined soybean oil (USP) is then added with continued mixing. The resulting solution is allowed to return to room temperature, then pumped through a 5 μm Meissner filter capsule.

Preferably, the pharmaceutical compositions of the present invention are filled into capsules at a desired dose, e.g., at a dose of 50 mg or 100 mg of the drug substance. Several different types of capsules may be used to manufacture the oral dosage forms of the present invention, e.g., gelatin capsules and non-gelatin capsules. Gelatin capsules are made of gelatin which is the product of the partial hydrolysis of collagen. The gelatin capsules can be employed as hard or soft gelatin capsules. Non-gelatin capsules may be made of carrageenan. Carrageenan is a natural polysaccharide hydrocolloid, which is derived from sea weed. Preferably, the oral dosage forms of the present invention are soft gelatin capsules. Additives may be added to the capsule shell including plasticizers, opacifiers, colorants, humectants, preservatives, flavorings, and buffering salts and acids. Colorants can be used for marketing and product identification/dose differentiation purposes. Suitable colorants include synthetic and natural dyes and combinations thereof. Optionally, the capsules can be film coated by employing film-coating agents conventional in the art. Preferably, the film-coating agent is an immediate release coating agent. Examples of immediate release coating agents include, but are not limited to, water soluble coating agents such as polyvinyl alcohol (PVA) and hypromellose (HPMC) based coating agents (available, e.g., from Coloron under the trade name OPADRY®). Alternatively, the capsules may be film coated by employing pH dependent enteric coating agents such as polymethacrylates (available, e.g., from Röhm under the trade name EUDRAGIT L 100-55®), hypromellose phthalate, hypromellose acetate succinate and cellulose acetate phthalate.

The oral dosage forms of the present invention comprising an allosteric adenosine A receptor enhancer, e.g., a 2-amino-3-aroylthiophene derivative, such as T-62, and at least one pharmaceutically acceptable lipid excipient in soft gelatin capsules, are stable over time such that the drug substance exhibits a pharmaceutically reasonable shelf life under standard storage conditions.

As illustrated herein in the Examples, the oral dosage forms of the present invention maintain the allosteric adenosine A₁ receptor enhancer, e.g., a 2-amino-3-aroylthiophene derivative, such as T-62, with a minimal degradation over time. Preferably, the oral dosage forms of the present inventions maintain at least 80% of the original amount of the allosteric adenosine A₁ receptor enhancer unchanged after about 3, about 6, about 9, about 12, about 18, about 24 and about 48 months. More preferably, at least about 85%, about 90% or about 95% of the original amount of the allosteric adenosine A₁ receptor enhancer is maintained unchanged after about 3, about 6, about 9, about 12, about 18, about 24 and about 48 months. It is preferred that the oral dosage forms of the present invention meet these stability parameters at an ambient temperature, e.g., at 25° C. and, preferably at high relative humidity (RH), e.g., 60% RH. More preferably, the oral dosage forms of the present invention meet these stability parameters at 30° C. and 65% RH and, most preferably, at 40° C. and 75% RH.

As to T-62 specifically, the present invention provides, an oral dosage form comprising T-62 and a pharmaceutically acceptable carrier medium as described herein above, wherein the oral dosage form exhibits an in vitro dissolution profile, when measured by the USP Basket Method at about 100 rpm in 900 mL of 0.05 M sodium phosphate buffer at about 37° C., such that after 10 min, from a mean of about 79% to a mean of about 92% (by weight) of T-62 is released, after 15 min, from a mean of about 84% to a mean of about 93% (by weight) of T-62 is released, after 30 min, from a mean of about 93% to a mean of about 98% (by weight) of T-62 is released, after 45 min, from a mean of about 94% to a mean of about 98% (by weight) of T-62 is released, after 60 min, from a mean of about 95% to a mean of about 98% (by weight) of T-62 is released, and after 90 min, from a mean of about 95% to a mean of about 98% (by weight) of T-62 is released.

Likewise, the present invention provides an oral dosage form comprising about 100 mg of T-62 and a pharmaceutically acceptable carrier medium as described herein above, said dosage form providing in man an arithmetic mean maximum plasma concentration of T-62 within the range of 80% to 125% of about 30 ng/mL at a median of about 2 hours following administration of a single dosage of said dosage form, whereby an arithmetic mean AUC₀₋₄₈ of T-62 is within the range of 80% to 125% of about 92 ng·h/mL.

Likewise, the present invention provides an oral dosage form comprising about 100 mg of T-62 and a pharmaceutically acceptable carrier medium as described herein above, said dosage form providing in man an arithmetic mean maximum plasma concentration of T-62 within the range of 80% to 125% of about 30 ng/mL at a median ranging from about 1 hour to about 2 hours following administration of a single dosage of said dosage form, whereby an arithmetic mean AUC_{0-inf} of T-62 is within the range of 80% to 125% of about 106 ng·h/mL.

Likewise, the present invention provides an oral dosage form comprising about 100 mg of T-62 and a pharmaceutically acceptable carrier medium as described herein above, said dosage form providing in man an arithmetic mean maximum plasma concentration of T-62 within the range of 80% to 125% of about 56 ng/mL at a median of about 1 hour following repeated administration of said dosage form every 12 hours through steady state conditions, whereby an arithmetic mean AUC_{0-τ} of T-62 is within the range of 80% to 125% of about 197 ng·h/mL.

Likewise, the present invention provides an oral dosage form comprising about 100 mg of T-62 and a pharmaceutically acceptable carrier medium as described herein above, said dosage form providing in man an arithmetic mean maximum plasma concentration of T-62 within the range of 80% to 125% of about 56 ng/mL at a median of about 1 hour following repeated administration of said dosage form every 12 hours through steady state conditions, whereby an arithmetic mean AUC_{0-inf} of T-62 is within the range of 80% to 125% of about 407 ng·h/mL.

Finally, the present invention relates to a method for the treatment of pain, including acute pain, e.g., postoperative pain, chronic pain, inflammatory pain, neuropathic pain and pain associated with migraine, in a subject, including man, in need thereof, which method comprises administering to the subject a pharmaceutical composition, or oral dosage forms thereof, comprising a therapeutically effective amount of an allosteric adenosine A₁ receptor enhancer, e.g., a 2-amino-3-aroylthiophene derivative, such as T-62, or a pharmaceutically acceptable salt thereof, and at least one pharmaceutically acceptable lipid excipient.

The therapeutically effective dosage of the allosteric adenosine A₁ receptor enhancer, e.g., a 2-amino-3-aroylthiophene derivative, such as T-62, can depend on a variety of factors, such as the specific compound to be administered, homeothermic species, age and/or the severity of the individual condition to be treated.

Preferred dosages for the allosteric adenosine A₁ receptor enhancers of the pharmaceutical compositions according to the present invention are therapeutically effective dosages. In general, however, doses employed for adult human treatment will typically be in the range of 0.02-5000 mg/day, preferably 1-1500 mg/day, e.g., for a patient of approximately 75 kg in weight. The desired dose may conveniently be presented in a single dose or as divided doses administered simultaneously or at appropriate intervals, for example as two, three, four or more sub-doses per day. For example, the doses of T-62 to be administered to subjects, including man, of approximately 75 kg body weight, especially the doses effective for enhancing the adenosine A₁ receptor function, e.g., to alleviate pain, are from about 1 mg to about 1000 mg, preferably from about 10 mg to about 800 mg/day. The daily dose may be divided between a daytime and night time dose. In a preferred embodiment of the present invention, the dosing regimen is once or twice per day. Since there is the potential of an allosteric adenosine A₁ receptor enhancer to cause sedation at a high dose, the higher doses are recommended for night use. For example, a dose ranging from about 50 to about 500 mg of T-62 in soft gelatin capsule form is recommended for daytime use while a dose ranging from about 600 to about 1000 mg is recommended as a nighttime dose. In a preferred embodiment of the present invention the dose employed for an adult human ranges from about 50 to about 800 mg/day.

The above description fully discloses the invention including preferred embodiments thereof. Modifications and improvements of the embodiments specifically disclosed herein are within the scope of the appended claims. Without further elaboration, it is believed that one skilled in the art can, using the preceding description, utilize the present invention to its fullest extent. Therefore, the Examples herein below are to be construed as merely illustrative of certain aspects of the present invention and are not a limitation of the scope of the present invention in any way.

### EXAMPLE 1

Composition 1: T-62 (C₁₅H₁₄NOSCl) was obtained from King Pharmaceuticals Research and Development, Inc. (Cary, N.C.) in dry powder form. T-62 was mixed using a mixer equipped with an OMNI 35 mm×195 mm probe in super-refined soybean oil (USP). The soybean oil was heated at 40° C.-50° C. during the homogenization step until a dosage of 10 or 20 mg/mL was obtained. The solution was sparged with Nitrogen throughout the process. The resulting solution was pumped through a 5 μm Meissner filter capsule, and placed in either soft gelatin capsules (Capsugel, Inc.), or into a 60-mL boston round, amber glass bottles with 20 mm-400 white child resistant caps (CRC) with foam liner cap.

Similarly, T-62 may be homogenized in corn oil (USP) until a dosage of 10 or 20 mg/mL is obtained, or alternatively in canola oil (USP) until a dosage of 25 mg/mL is obtained.

TABLE 1
Composition 1

| | | |
|---|---|---|
| Ingredient | % w/w | Amount/Batch (g) |

T-62 Solution - 10 mg/mL

| | | |
|---|---|---|
| T-62 | 1.1 | 81.6 |
| Super Refined Soybean Oil | 98.9 | 7336.4 |
| Total | 100.0 | 7418.0 |

T-62 Solution - 20 mg/mL

| | | |
|---|---|---|
| T-62 | 2.2 | 165 |
| Super Refined Soybean Oil | 97.8 | 7334 |
| Total | 100.0 | 7499 |

### EXAMPLE 2

Composition 2: T-62 (C₁₅H₁₄NOSCl) may be obtained from King Pharmaceuticals (Cary, N.C.) in dry powder form. T-62 was screened through a #40 screen and then added to a mixture of propylene glycol monocaprylate (CAPRYOL 90®), caprylocaproyl macrogol-8 glycerides (LABRASOL®), super refined soybean oil (USP) and polysorbate 80 (CRILLET 4 HP®) at 50° C. (±5° C.). The solution was mixed with a propeller mixer to dissolve the T-62. The solution was sparged with Nitrogen throughout the process. The solution was pumped through a 5 μm Meissner filter capsule, and had a density of 1.006 g/mL at 25° C.

Oral Dosage Form 2: The resulting solution was encapsulated into hypromellose (HPMC) capsules at a 30 mg dosage strength, optionally contained within Enterion™ capsule.

TABLE 2
Composition and Oral Dosage Form 2

| Ingredient | % w/w | mg/cap |
|---|---|---|
| T-62 | 6.08 | 30.4 |
| propylene glycol monocaprylate (CAPRYOL 90 ®) | 43.92 | 219.6 |
| caprylocaproyl macrogol-8 glycerides | 16.70 | 83.5 |
| (LABRASOL ®) | | |
| super refined soybean oil (USP) | 25.00 | 125.0 |
| polysorbate 80 (CRILLET 4 HP ®) | 8.30 | 41.5 |
| TOTAL | 100.00 | 500.0 |

### EXAMPLE 3

Composition 3: T-62 (C₁₅H₁₄NOSCl) may be obtained from King Pharmaceuticals (Cary, N.C.). T-62 was milled using a Quadro Comil 197 with screen 2A018R01530 and impeller 2A16011730212 at 2400 rpm. The milled T-62 was then added to a mixture of super refined soybean oil (USP) with propylene glycol monocaprylate (CAPRYOL 90®), caprylocaproyl macrogol-8 glycerides (LABRASOL®), and polysorbate 80 (CRILLET 4 HP®) heated to 50-55° C. The solution was sparged with Nitrogen throughout the process. The T-62 was mixed until dissolved, then pumped through a 5 μm Meissner filter capsule.

Oral Dosage Form 3: The resulting solution may be encapsulated into hard gelatin capsules (Capsugel, Inc.) at a 70 mg dosage strength.

TABLE 3
Composition and Oral Dosage Form 3

| Ingredient | % w/w | mg/cap |
|---|---|---|
| T-62 | 8.33 | 70.4 |
| propylene glycol monocaprylate (CAPRYOL 90 ®) | 41.67 | 352.1 |
| caprylocaproyl macrogol-8 glycerides | 16.70 | 141.1 |
| (LABRASOL ®) | | |
| super refined soybean oil (USP) | 25.00 | 211.3 |
| polysorbate 80 (CRILLET 4 HP ®) | 8.30 | 70.1 |
| TOTAL | 100.00 | 845.0 |

### EXAMPLE 4

Composition 4: T-62 (C₁₅H₁₄NOSCl) may be obtained from King Pharmaceuticals (Cary, N.C.). T-62 was milled using a Quadro Comil 197 with screen 2A018R01530 and impeller 2A16011730212 at 2400 rpm. The milled T-62 was then added to a mixture of super refined soybean oil (USP) with propylene glycol monocaprylate (CAPRYOL 90®), caprylocaproyl macrogol-8 glycerides (LABRASOL®), and polysorbate 80 (CRILLET 4 HP®) heated to 50° C.-55° C. The solution was sparged with Nitrogen throughout the process. The T-62 was mixed until dissolved, then pumped through a 5 μm Meissner filter capsule.

Oral Dosage Form 4: The resulting solution may be encapsulated into soft elastic gelatin (SEG) capsules (Capsugel, Inc.) at a 100 mg dosage strength. The SEG capsules may be optionally enteric coated with Eudragit L 100-55 (Rohm).

TABLE 4
Composition and Oral Dosage Form 4

| Ingredient | % w/w | mg/cap |
|---|---|---|
| T-62 | 8.33 | 100.0 |
| propylene glycol monocaprylate (CAPRYOL 90 ®) | 41.67 | 500.0 |
| caprylocaproyl macrogol-8 glycerides | 16.70 | 200.4 |
| (LABRASOL ®) | | |
| super refined soybean oil (USP) | 25.00 | 300.0 |
| polysorbate 80 (CRILLET 4 HP ®) | 8.30 | 99.6 |
| TOTAL | 100.00 | 1200.0 |

### EXAMPLE 5

Composition 5: T-62 (C₁₅H₁₄NOSCl) may be obtained from King Pharmaceuticals (Cary, N.C.). T-62 was milled using a Quadro Comil 197 with screen 2A018R01530 and impeller 2A16011730212 at 2400 rpm. The milled T-62 was then added to a mixture of super refined soybean oil (USP) with propylene glycol monocaprylate (CAPRYOL 90®), caprylocaproyl macrogol-8 glycerides (LABRASOL®), and polysorbate 80 (CRILLET 4 HP®) heated to 50° C.-55° C. The solution was sparged with Nitrogen throughout the process. The T-62 was mixed until dissolved, then pumped through a 5 μm Meissner filter capsule.

Oral Dosage Form 5: The resulting solution may be encapsulated into SEG capsules at a 50 mg dosage strength.

TABLE 5
Composition and Oral Dosage Form 5

| Ingredient | % w/w | mg/cap |
|---|---|---|
| T-62 | 4.17 | 50.0 |
| propylene glycol monocaprylate (CAPRYOL 90 ®) | 45.83 | 550.0 |
| caprylocaproyl macrogol-8 glycerides | 16.70 | 200.4 |
| (LABRASOL ®) | | |
| super refined soybean oil (USP) | 25.00 | 300.0 |
| polysorbate 80 (CRILLET 4 HP ®) | 8.30 | 99.6 |
| TOTAL | 100.00 | 1200.0 |

### EXAMPLE 6

Composition 6: T-62 (C₁₅H₁₄NOSCl) may be obtained from King Pharmaceuticals (Cary, N.C.). T-62 was milled using a Quadro Comil 197 with screen 2A018R01530 and impeller 2A16011730212 at 2400 rpm. The milled T-62 was then micronized using a Glen Mills Jet Mill with Nitrogen as the propellant. The T-62 was passed through the Jet Mill twice to reduce the particle size to a mean diameter of 12.2 μm. The micronized T-62 was then mixed into a mixture of super refined soybean oil (USP) with propylene glycol monocaprylate (CAPRYOL 90®), caprylocaproyl macrogol-8 glycerides (LABRASOL®), and polysorbate 80 (CRILLET 4 HP®) using a propeller type mixer to incorporate the T-62. The mixture was sparged with Nitrogen throughout the process. The resulting solution was pumped through a 5 μm Meissner filter capsule.

Oral Dosage Form 6: The resulting solution was encapsulated into hard gelatin capsules (size 00 Capsules, obtained from Capsugel Inc.) at a 70 mg dosage strength.

TABLE 6
Composition and Oral Dosage Form 6

| Ingredient | % w/w | mg/cap |
|---|---|---|
| T-62 | 8.33 | 70.4 |
| propylene glycol monocaprylate (CAPRYOL 90 ®) | 41.67 | 352.1 |
| caprylocaproyl macrogol-8 glycerides | 16.70 | 141.1 |
| (LABRASOL ®) | | |
| super refined soybean oil (USP) | 25.00 | 211.3 |
| polysorbate 80 (CRILLET 4 HP ®) | 8.30 | 70.1 |
| TOTAL | 100.00 | 845.0 |

### EXAMPLE 7

Composition 7: T-62 (C₁₅H₁₄NOSCl) was obtained from Cambrex, Inc. T-62 was milled using a Quadro Comil 197 with screen 2A018R01530 and impeller 2A16011730212 at 2400 rpm. The milled T-62 was then added to a mixture of propylene glycol monocaprylate (CAPRYOL 90®), caprylocaproyl macrogol-8 glycerides (LABRASOL®), and polysorbate 80 (CRILLET 4 HP®) at 45° C.±5° C. The T-62 was mixed with a propeller mixer and the solution was sparged with Nitrogen throughout the process. Super refined soybean oil was added with continued mixing. The composition was allowed to return to room temperature, then pumped through a 5 μm Meissner filter capsule.

Oral Dosage Form 7: The resulting solution was encapsulated into SEG capsules at a 100 mg dosage strength.

TABLE 7
Composition and Oral Dosage Form 7

| Ingredient | % w/w | mg/cap |
|---|---|---|
| T-62 | 8.33 | 100.0 |
| propylene glycol monocaprylate (CAPRYOL 90 ®) | 41.67 | 500.0 |
| caprylocaproyl macrogol-8 glycerides | 29.17 | 200.4 |
| (LABRASOL ®) | | |
| super refined soybean oil (USP) | 12.50 | 300.0 |
| polysorbate 80 (CRILLET 4 HP ®) | 8.33 | 99.4 |
| TOTAL | 100.00 | 1200.0 |

Optionally, the capsules may be film coated, e.g., by OPADRY® II film coating system. The coating suspension may be prepared, e.g., by adding 100 g of OPADRY® II White powder to a mixture of 405 g of water and 495 g of absolute ethanol while mixing at a speed capable of producing and maintaining a vortex. After all OPADRY® II powder has been added, the speed is reduced to nearly eliminate the vortex, and the mixing is then continued for 45 min further. The resulting OPADRY® II dispersion is agitated gently during the coating process. The OPADRY® II dispersion may be applied to a coating weight gain between 3% to 5%.

### EXAMPLE 8

Composition 8: T-62 (C₁₅H₁₄NOSCl) was obtained from Cambrex, Inc. T-62 was milled using a Quadro Comil 197 with screen 2A018R01530 and impeller 2A16011730212 at 2400 rpm. The milled T-62 was then added to a mixture of propylene glycol monocaprylate (CAPRYOL 90®), caprylocaproyl macrogol-8 glycerides (LABRASOL®), polysorbate 80 (CRILLET 4 HP®) and ethanol at 45° C.±5° C. The T-62 was mixed with a propeller mixer and the solution was sparged with Nitrogen throughout the process. Super refined soybean oil was added with continued mixing until the T-62 was dissolved. The composition was allowed to return to room temperature, then pumped through a 5 μm Meissner filter capsule.

Oral Dosage Form 8: The resulting solution was encapsulated into soft elastic gelatin capsules at 100 mg dosage strength.

TABLE 8
Composition and Oral Dosage Form 8

| Ingredient | % w/w | mg/cap |
|---|---|---|
| T-62 | 8.33 | 100.0 |
| propylene glycol monocaprylate (CAPRYOL 90 ®) | 41.67 | 500.0 |
| caprylocaproyl macrogol-8 glycerides | 21.17 | 254.0 |
| (LABRASOL ®) | | |
| super refined soybean oil (USP) | 12.50 | 150.0 |
| polysorbate 80 (CRILLET 4 HP ®) | 8.33 | 100.0 |
| Ethanol | 8.00 | 96.0 |
| TOTAL | 100.00 | 1200.0 |

### EXAMPLE 9

Oral dosage forms 4, 5, 7 and 8 were tested for the stability of T-62 at 25° C. at 60% relative humidity (RH); at 30° C. and 65% RH; and/or at 40° C. and 75% RH; contained in high-density polyethylene (HDPE) bottles sealed with CRC caps. The dosage forms were tested at different time points, and the quantity of T-62 was determined by HPLC analysis using an Agilent HPLC system equipped with a dual wavelength photodiode array detector and a Zorbax SB-C₁₈ column (150 mm×4.6 mm, 5 μm). The results are shown in Tables 9, 10, 11 and 12 (expressed as a percentage of T-62 of the label claim which is the amount of the drug substance in the particular dosage form).

### Sample Preparation:

The HPLC samples were prepared by placing 10 uncut SEG capsules together with a stir bar into an appropriately sized volumetric flask (1000 mL flask for 50 mg capsules and 2000 mL flask for 100 mg capsules). The flask was then filled to approximately half volume with a 3:2-mixture of acetonitrile (ACN) and deionized water (DI H₂O). The preparation was stirred for 2 hours, and the stir bar was removed. The preparation was diluted to full volume with a 3:2-mixture of ACN and DI H₂O, and sonicated for 15 min. The preparation was then filtered through a 0.45 μm Nylon filter, and the first 3 mL were discarded. An aliquot of 6 mL was transferred into a 150 mL volumetric flask and diluted to full volume with a 3:2-mixture of ACN and DI H₂O.

### HPLC Analysis:

Flow Rate: 1.0 mL/min.

Injection Volume: 50 μL.

Column Temperature: 50° C.

Detector Wavelength: 245 nm.

Run Time: 60 min.

Retention Time for T-62: about 16 min.

Mobile Phase A: a 10:90 mixture of ACN and 20 mM KH₂PO₄, pH 2.5.

Mobile Phase B: a 90:8:2 mixture of ACN, DI H₂O and 20 mM KH₂PO₄, pH 2.5.

TABLE 9
Stability of Oral Dosage Form 4

| | T-62: % Label Claim | | |
|---|---|---|---|
| | Conditions for Stability Testing | | |
| | | | 40° C. & 75% |
| | 25° C. & 60% RH | 30° C. & 65% RH | RH |
| Sample 1 | | | |
| Initial | 93.1% | 93.1% | 93.1% |
| 1 month | | | 93.8% |
| 3 months | 93.4% | | |
| 6 months | 94.8% | | 93.0% |
| 9 months | 93.5% | 93.4% | |
| 12 months | 94.4% | 93.3% | |
| Sample 2 | | | |
| Initial | 98.8% | | 98.8% |
| 1 month | | | 94.7% |
| 2 months | | | 92.2% |
| 3 months | 94.2% | | 97.2% |
| 6 months | 97.1% | | 99.4% |

TABLE 10
Stability of Oral Dosage Form 5

| | T-62: % Label Claim | | |
|---|---|---|---|
| | Conditions for Stability Testing | | |
| | | | 40° C. & 75% |
| Sample | 25° C. & 60% RH | 30° C. & 65% RH | RH |
| Initial | 96.5% | 96.5% | 96.5% |
| 1 month | | | 96.8% |
| 2 months | | | 95.4% |
| 3 months | 95.7% | | 96.2% |
| 6 months | 98.0% | 96.6% | 94.7% |

TABLE 11
Stability of Oral Dosage Form 7

| | T-62: % Label Claim | | |
|---|---|---|---|
| | Conditions for Stability Testing | | |
| | | | 40° C. & 75% |
| Sample | 25° C. & 60% RH | 30° C. & 65% RH | RH |
| Initial | 95.4% | 95.4% | 95.4% |
| 1 month | | | 96.9% |
| 2 months | | | 96.1% |
| 3 months | | 95.8% | 98.4% |
| 6 months | 95.4% | 95.4% | 96.8% |
| 9 months | 93.7% | 94.2% | |
| 12 months | 94.2% | 93.1% | |

TABLE 12
Stability of Oral Dosage Form 8

T-62: % Label Claim
Conditions for Stability Testing

| Sample | 25° C. & 60% RH | 40° C. & 75% RH |
|---|---|---|
| Initial | 93.0% | 93.0% |
| 1 month | | 97.3% |
| 2 months | | 96.0% |
| 3 months | | 94.4% |
| 6 months | 95.4% | 94.7% |

### EXAMPLE 10

### Dissolution Method

In vitro dissolution studies were conducted on oral dosage forms 4, 5, 7 and 8 by employing the current USP Basket Method <711> under the following dissolution conditions. The results are shown in Table 13.

TABLE 13

| | % of T-62 Dissolved^{a} | | | |
|---|---|---|---|---|
| Time | Composition 4 | Composition 5 | Composition 7 | Composition 8 |
| (min) | (100 mg) | (50 mg) | (100 mg) | (100 mg) |
| 10 | 83.0 | 79.4 | 91.7 | 87.1 |
| 15 | 84.1 | 84.9 | 93.3 | 88.6 |
| 30 | 93.2 | 93.6 | 97.9 | 94.9 |
| 45 | 97.0 | 94.0 | 97.8 | 95.1 |
| 60 | 97.3 | 94.6 | 97.6 | 96.0 |
| 90 | 98.3 | 95.0 | 97.9 | 95.5 |

^{a}an arithmetic mean of 6 experiments.

Apparatus: VanKel Model VK7000 Dissolution Bath, Apparatus I (Baskets).

Dissolution Medium: 0.05 M sodium phosphate buffer pH 6.8 with 1% of hexadecyltrimethylammonium bromide.

Dissolution Medium Volume: 900 mL.

Temperature: 37° C.±0.5° C.

Rotation Speed: 100 rpm.

Sample Size: 1 capsule per basket.

Sampling Time: 10, 15, 30, 45, 60 and 90 min.

Sampling Volume: 1 mL.

### HPLC Analysis

Column: Agilent Zorbax SB-C₁₈ column, 150 mm×4.6 mm, 5 μm.

Mobile Phase: a 20:80 mixture of acidified water (pH 2.5, phosphoric acid) and ACN.

Flow Rate: 1 mL/min.

Injection Volume: 10 μL.

Column Temperature: 50° C.

Detector Wavelength: 366 nm.

Run Time: 7 min.

Retention Time for T-62: about 3 min.

### EXAMPLE 11

A single-center, 3-part study: Parts A and B were randomized, double-blind, and placebo-controlled, and evaluated the safety, tolerability, and pharmacokinetics of single, escalating dose levels of a T-62 composition in soft gelatin capsules (oral dosage form 4) in young healthy subjects (Part A), and a single dose of a T-62 composition in soft gelatin capsules (oral dosage form 4) in elderly healthy subjects (Part B). In Part C, the effect of food on the bioavailability of a single dose of T-62 (oral dosage form 4) in young healthy subjects was evaluated in an open label, randomized, crossover fashion.

Part A: The dose escalation phase of the study consisted of 6 cohorts of 12 young (18-45 years of age) healthy volunteers, a total of 72 subjects, randomly assigned in a 3:1 allocation to receive a single dose of either T-62 or placebo under fasted conditions. For all cohorts, subjects fasted for a minimum of 7 hours pre-dose to 4 hours post-dose. Subjects in the first cohort received a single dose of 100 mg of T-62 (n=9) or placebo (n=3). Subsequent cohorts of 12 new subjects received placebo or a higher dose level of T-62: Nine subjects in each cohort received a total of 2×100, 4×100, 8×100, 10×100 or 12×100 mg capsules of T-62; three subjects per dose level received placebo.

Part B: A total of 15 elderly subjects (≧65 years of age) were randomly assigned in a 4:1 allocation to receive a single dose of 4×100 mg of T-62 (n=12) or placebo (n=3) under fasted conditions, i.e., subjects were fasted for a minimum of 7 hours pre-dose to 4 hours post-dose.

Part C: A single cohort of 16 young (18-45 years of age) healthy volunteers were enrolled to evaluate the effect of food on the bioavailability and pharmacokinetics of a single-dose of 4×100 mg soft gelatin capsules of T-62. Subjects were randomly assigned in a 1:1 allocation to 1 of 2 treatment sequences (i.e., fed/fasted or fasted/fed) in a crossover fashion.

Each subject in Parts A and B completed Screening, Baseline, Treatment, and Follow-Up Phases. The Screening Phase was conducted on an outpatient basis within 30 days, but no less than 3 days, prior to the start of the Baseline Phase. The Baseline Phase consisted of clinical research unit (CRU) admission and final qualification assessments. The Treatment Phase was comprised of dosing, post-treatment safety assessments, and blood collections. Subjects were discharged approximately 50 hours after study drug administration on Day 3. The Follow-Up Phase occurred 2 to 4 days after discharge from the CRU.

Each subject in Part C completed a Screening Phase, Baseline and Treatment Phases for both crossover Dosing Periods I and II, and a single Follow-Up Phase. The Screening Phase was conducted on an outpatient basis within 30 days, but no less than 3 days, prior to the start of the Baseline Phase for Dosing Period I. Each Baseline Phase consisted of CRU admission and final/continuing qualification assessments. Each Treatment Phase was comprised of dosing, post-treatment safety assessments, and blood collections. T-62 was administered and post-treatment assessments were conducted on Day 1 of Dosing Period I. Subjects were discharged approximately 50 hours after study drug administration on Day 3. Following a 3-day washout, subjects crossed over and entered Dosing Period II. Subjects were re-admitted to the CRU one day before T-62 administration in Dosing Period II for Baseline assessments. T-62 was be administered and post-treatment assessments were conducted on Day 1 of Dosing Period II. Subjects were discharged approximately 50 hours after study drug administration on Day 3. The Follow-Up Phase occurred 2 to 4 days after discharge from the CRU following Dosing Period II. For each Dosing period, subjects randomized to receive T-62 under fed conditions were given a high fat breakfast on Day 1 about half an hour prior to dosing. Fasting subjects were not allowed to eat any food beginning a minimum of 7 hours pre-dose to 4 hours post-dose.

Blood samples for determining plasma concentrations of T-62 were obtained immediately prior to dosing and at regular intervals post-dose over 48 hours period after the dose in each treatment cohort.

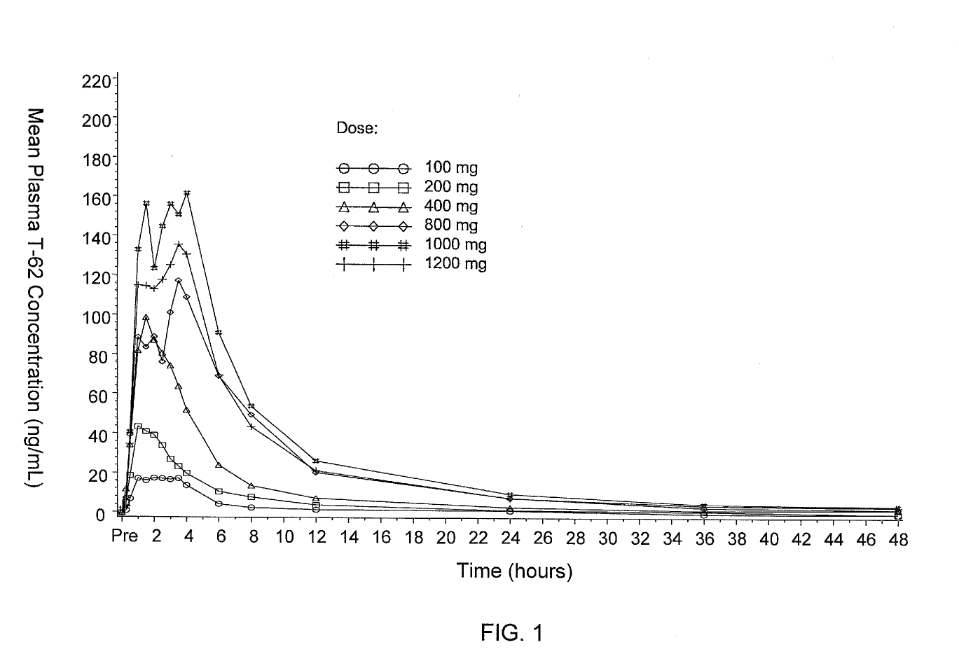

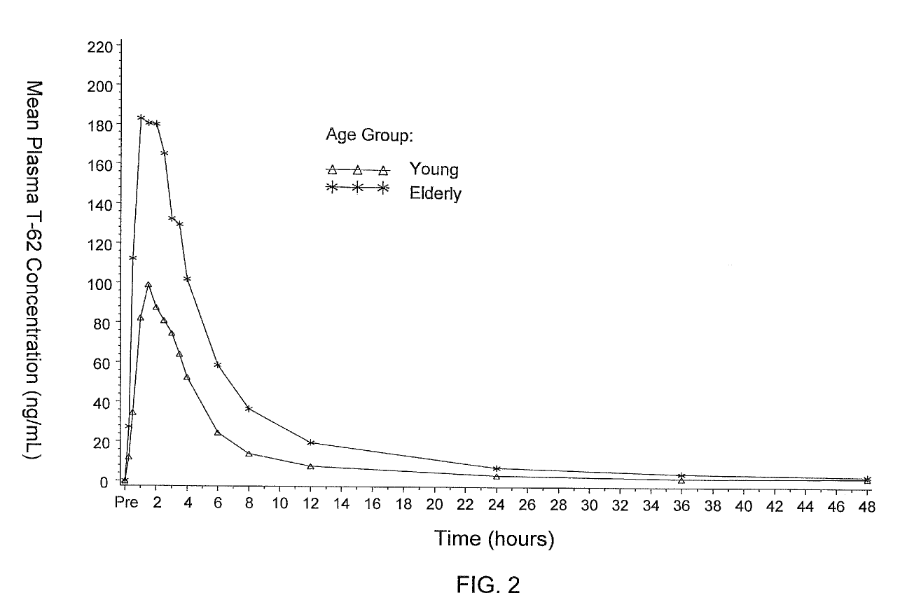

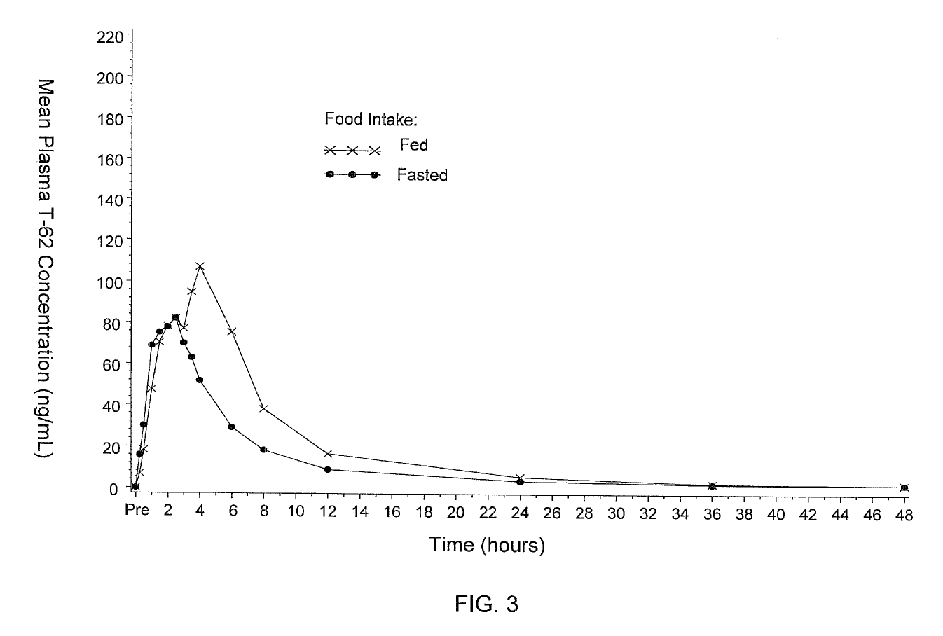

Plasma concentrations of T-62 were used to determine the pharmacokinetic parameters using non-compartmental methods, and the data are summarized in FIG. 1-3 and Table 14.

TABLE 14
Arithmetic Means (±SD) for Selected Pharmacokinetic
Parameters of T-62 by Dose Group

| | T-62 Dose | Tₘₐₓ | | AUC₀₋₄₈ | AUC_{0-inf} |
|---|---|---|---|---|---|
| Cohort | (mg) | (h)^{e} | Cₘₐₓ (ng/mL) | (hr * ng/mL) | (hr * ng/mL) |
| Part A^{a} | 100 | 2.0 | 30.0 (15.1) | 92.2 (39.9) | 106 (45.9) |
| Part A^{a} | 2 × 100 | 1.0 | 47.7 (26.6) | 224 (89.7) | 242 (90.8) |
| Part A^{a} | 4 × 100 | 2.0 | 110 (72.6) | 525 (304) | 557 (318) |
| Part A^{a} | 8 × 100 | 3.5 | 165 (114) | 978 (489) | 1018 (508) |
| Part A^{a} | 10 × 100 | 3.0 | 213 (97.3) | 1367 (424) | 1459 (453) |
| Part A^{a} | 12 × 100 | 3.5 | 157 (84.5) | 1114 (598) | 1204 (688) |
| Part B^{b} | 4 × 100 | 1.8 | 227 (118) | 1195 (471) | 1288 (500) |
| Part C, | 4 × 100 | 3.5 | 154 (65.3) | 884 (248) | 933 (243) |
| Fed^{c} | | | | | |
| Part C, | 4 × 100 | 2.3 | 95.8 (45.1) | 533 (219) | 576 (227) |
| Fasted^{d} | | | | | |

^{a}n = 9,
^{b}n = 12,
^{c}n = 16, and
^{d}n = 14;
^{e}Median.

### EXAMPLE 12

A single-center, randomized, double-blind, parallel-group, placebo-controlled study of the safety, tolerability, and pharmacokinetics of escalating multiple doses of a T-62 composition in soft gelatin capsules (oral dosage form 4) in healthy adult male and female subjects was carried out.

Three cohorts of 12 subjects were enrolled for the study (19-38 years of age). Subjects in each cohort were randomly assigned in a 3:1 allocation to receive multiple doses of either T-62 (n=9) or placebo (n=3). Subjects in each cohort received study medication for a total of 6 days. Subjects in the first cohort received multiple doses of 100 mg T-62 or placebo. Subsequent cohorts of 12 new subjects each received multiple doses of placebo or a higher dose level of T-62 (i.e., 2×100 mg for the second cohort and 4×100 mg for the third cohort).

Each subject in each dosing cohort completed Screening, Baseline, Treatment, and Follow-Up Phases. The Screening Phase was conducted on an outpatient basis within 30 days, but no less than 3 days, prior to the start of the Baseline Phase. The Baseline Phase consisted of clinical research unit (CRU) admission and final qualification assessments. The Treatment Phase comprised of dosing, post-treatment safety assessments, and blood collection.

On the morning of Day 1, subjects received a single dose of study drug; no additional study drug were administered on Day 1. Twice-daily dosing (one morning dose and one evening dose) commenced on Day 2 and continue through Day 5. Subjects received a final dose of study drug on the morning of Day 6. Once subjects in each cohort had completed dosing, an additional 48 hours of blood sampling was conducted following the final dose to characterize the pharmacokinetics of T-62 at steady state. Subjects were discharged at the end of the 48-hour blood sampling (Day 8). The Follow-Up Phase occurred 2 days (but no more than 4 days) after discharge from the CRU.

For all cohorts, on Day 2 through Day 5, subjects were not allowed to eat any food beginning 1 hour prior to the morning and evening doses of study drug and until 2 hours after study drug administration. On Day 1 and Day 6, subjects fasted for a minimum of 9 hours pre-dose to 4 hours post-dose.

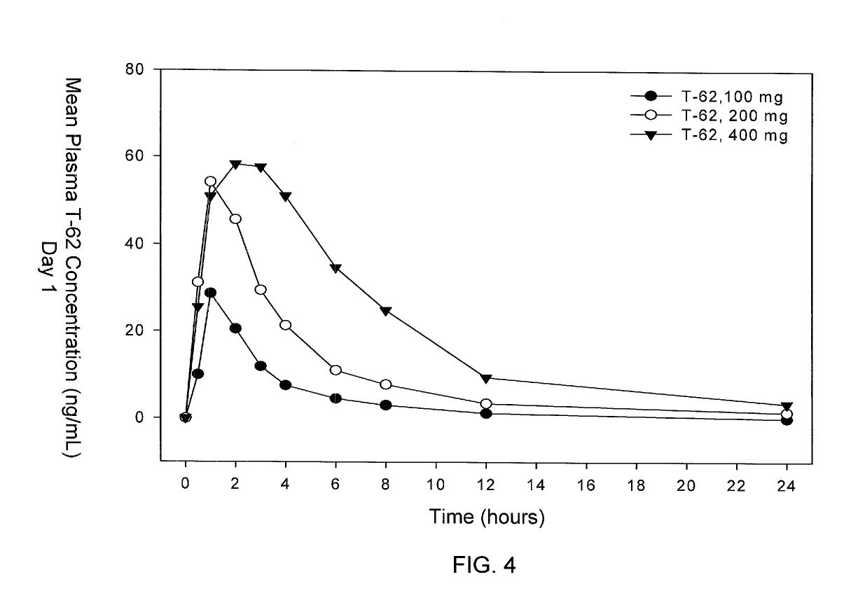

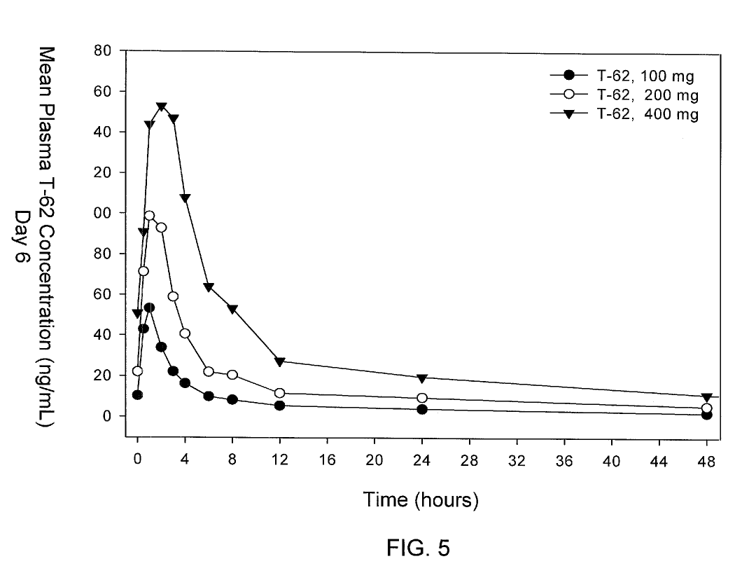

Blood samples for determining plasma concentrations of T-62 and pharmacokinetic parameters were obtained immediately prior to dosing on Day 1 and at regular intervals post-dosing over 12 hours in each treatment cohort. On Days 2-5, blood samples for pharmacokinetic analysis were collected each day prior to the morning and evening doses of study drug. Once subjects in each cohort had completed dosing on Day 6, an additional 48 hours of plasma sampling were conducted following the final dose of study drug to characterize the pharmacokinetics of T-62 at steady state. The results are depicted in FIGS. 4 and 5, and summarized in Tables 15 and 16.

TABLE 15
Arithmetic Means (±SD) for Selected Pharmacokinetic Parameters after
Administration of a Single Dose of T-62 (Day 1) by Dose Group

| T-62 Dose | | Cₘₐₓ | AUC_{0-τ} | AUC_{0-inf} |
|---|---|---|---|---|
| (mg) | Tₘₐₓ (h)^{a} | (ng/mL) | (hr * ng/mL)^{b} | (hr * ng/mL) |
| 100 (n = 8) | 1.0 | 28.8 (9.30) | 89.5 (31.6) | 96.9 (33.4) |
| 2 × 100 (n = 9) | 1.0 | 60.7 (29.2) | 211 (83.9) | 254 (99.1) |
| 4 × 100 (n = 9) | 2.0 | 71.4 (33.3) | 399 (168) | 496 (181) |

^{a}Median;
^{b}τ = dosing interval, 12 hours.

TABLE 16
Arithmetic Means (±SD) for Selected Pharmacokinetic
Parameters of T-62 at Steady State (Day 6) by Dose Group

| T-62 Dose | | Cₘₐₓ | AUC_{0-τ} | AUC_{0-inf} |
|---|---|---|---|---|
| (mg, BID) | Tₘₐₓ (h)^{a} | (ng/mL) | (hr * ng/mL)^{b} | (hr * ng/mL) |
| 100 (n = 8) | 1.0 | 55.9 (22.5) | 197 (64.5) | 407 (150) |
| 2 × 100 (n = 9) | 1.0 | 107 (37.2) | 450 (176) | 995 (482) |
| 4 × 100 (n = 9) | 2.0 | 179 (78.7) | 958 (358) | 2210 (901) |

^{a}Median;
^{b}τ = dosing interval, 12 hours.

What is claimed is:

1. A pharmaceutical composition in the form of a solution comprising a therapeutically effective amount of a 2-amino-3-aroylthiophene derived allosteric adenosine A₁ receptor enhancer, or a pharmaceutically acceptable salt thereof, and at least one pharmaceutically acceptable lipid excipient, wherein the lipid excipient is present in an amount of more than about 5% by weight based on the total weight of the pharmaceutical composition.

2. A pharmaceutical composition according to claim 1, wherein the pharmaceutical composition further comprises one or more surfactants.

3. A pharmaceutical composition according to claim 2, wherein the pharmaceutical composition is self-microemulsifying.

4. A pharmaceutical composition according to claim 3, wherein the 2-amino-3-aroylthiophene derived allosteric adenosine A₁ receptor enhancer is selected from the group of compounds of the formulae 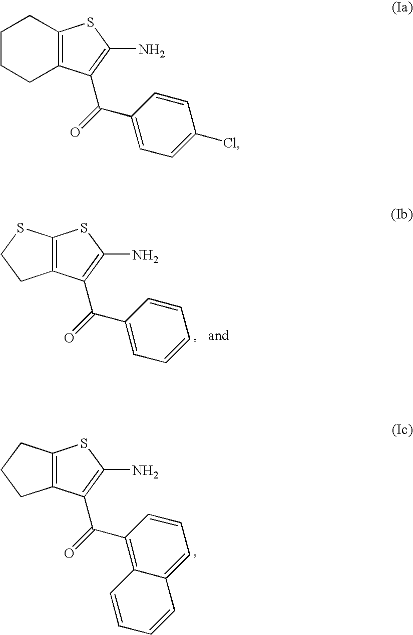
or in each case, a pharmaceutically acceptable salt thereof.

5. A pharmaceutical composition according to claim 4, wherein the lipid excipient is soybean oil.

6. A pharmaceutical composition according to claim 5, wherein soybean oil is present in an amount ranging from about 12% to about 25% by weight based on the total weight of the pharmaceutical composition.

7. A pharmaceutical composition according to claim 6, wherein the one or more surfactants are selected from the group consisting of propylene glycol monocaprylate, caprylocaproyl macrogol-8 glycerides and polysorbate 80.

8. A pharmaceutical composition according to claim 7, wherein the one or more surfactants are present in an amount ranging from about 65% to about 85% by weight based on the total weight of the pharmaceutical composition.

9. A pharmaceutical composition according to claim 8, wherein the 2-amino-3-aroylthiophene derived allosteric adenosine A₁ receptor enhancer is the compound of the formula

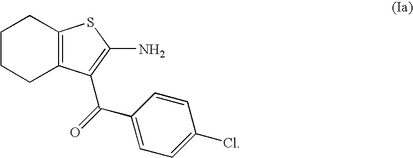

10. A pharmaceutical composition according to claim 9, wherein the compound of formula (Ia) is present in an amount ranging from about 4% to about 9% by weight based on the total weight of the pharmaceutical composition.

11. A pharmaceutical composition according to claim 10, wherein the composition is filled into soft gelatin capsules to provide an oral dosage form.

12. A pharmaceutical composition according to claim 11, wherein the oral dosage form maintains at least 80% of the original amount of the compound of formula (Ia) unchanged up to and including 48 months.

13. An oral dosage form comprising from about 50 mg to about 100 mg of T-62 and a pharmaceutically acceptable carrier medium, wherein the oral dosage form exhibits an in vitro dissolution profile, when measured by the USP Basket Method at about 100 rpm in 900 mL of 0.05 M sodium phosphate buffer at about 37° C., such that after 10 min, from a mean of about 79% to a mean of about 92% (by weight) of T-62 is released, after 15 min, from a mean of about 84% to a mean of about 93% (by weight) of T-62 is released, after 30 min, from a mean of about 93% to a mean of about 98% (by weight) of T-62 is released, after 45 min, from a mean of about 94% to a mean of about 98% (by weight) of T-62 is released, after 60 min, from a mean of about 95% to a mean of about 98% (by weight) of T-62 is released, and after 90 min, from a mean of about 95% to a mean of about 98% (by weight) of T-62 is released.

14. An oral dosage form according to claims 13, wherein the carrier medium comprises at least one lipid excipient.

15. An oral dosage form according to claim 14, wherein the carrier medium further comprises one or more surfactants.

16. An oral dosage form according to claims 15, wherein the carrier medium is self-microemulsifying.

17. An oral dosage form according to claim 16, wherein the lipid excipient is soybean oil.

18. An oral dosage form according to claim 17, wherein the one or more surfactants are selected from the group consisting of propylene glycol monocaprylate, caprylocaproyl macrogol-8 glycerides and polysorbate 80.

19. An oral dosage form comprising about 100 mg of T-62 and a pharmaceutically acceptable carrier medium, said dosage form providing in man a mean maximum plasma concentration of T-62 within the range of 80% to 125% of about 30 ng/mL at a median of about 2 hours following administration of a single dosage of said dosage form, whereby an arithmetic mean AUC₀₋₄₈ of T-62 is within the range of 80% to 125% of about 92 ng·h/mL.

20. An oral dosage form according to claims 19, wherein the carrier medium comprises at least one lipid excipient.

21. An oral dosage form according to claim 20, wherein the carrier medium further comprises one or more surfactants.

22. An oral dosage form according to claims 21, wherein the carrier medium is self-microemulsifying.

23. An oral dosage form according to claim 22, wherein the lipid excipient is soybean oil.

24. An oral dosage form according to claim 23, wherein the one or more surfactants are selected from the group consisting of propylene glycol monocaprylate, caprylocaproyl macrogol-8 glycerides and polysorbate 80.

25. An oral dosage form comprising about 100 mg of T-62 and a pharmaceutically acceptable carrier medium, said dosage form providing in man an arithmetic mean maximum plasma concentration of T-62 within the range of 80% to 125% of about 30 ng/mL at a median ranging from about 1 hour to about 2 hours following administration of a single dosage of said dosage form, whereby an arithmetic mean AUC_{0-inf} of T-62 is within the range of 80% to 125% of about 106 ng·h/mL.

26. An oral dosage form according to claims 25, wherein the carrier medium comprises at least one lipid excipient.

27. An oral dosage form according to claim 26, wherein the carrier medium further comprises one or more surfactants.

28. An oral dosage form according to claims 27, wherein the carrier medium is self-microemulsifying.

29. An oral dosage form according to claim 28, wherein the lipid excipient is soybean oil.

30. An oral dosage form according to claim 29, wherein the one or more surfactants are selected from the group consisting of propylene glycol monocaprylate, caprylocaproyl macrogol-8 glycerides and polysorbate 80.

31. An oral dosage form comprising about 100 mg of T-62 and a pharmaceutically acceptable carrier medium, said dosage form providing in man an arithmetic mean maximum plasma concentration of T-62 within the range of 80% to 125% of about 56 ng/mL at a median of about 1 hour following repeated administration of said dosage form every 12 hours through steady state conditions, whereby an arithmetic mean AUC_{0-τ} of T-62 is within the range of 80% to 125% of about 197 ng·h/mL.

32. An oral dosage form according to claims 31, wherein the carrier medium comprises at least one lipid excipient.

33. An oral dosage form according to claim 32, wherein the carrier medium further comprises one or more surfactants.

34. An oral dosage form according to claims 33, wherein the carrier medium is self-microemulsifying.

35. An oral dosage form according to claim 34, wherein the lipid excipient is soybean oil.

36. An oral dosage form according to claim 35, wherein the one or more surfactants are selected from the group consisting of propylene glycol monocaprylate, caprylocaproyl macrogol-8 glycerides and polysorbate 80.

37. An oral dosage form comprising about 100 mg of T-62 and a pharmaceutically acceptable carrier medium, said dosage form providing in man an arithmetic mean maximum plasma concentration of T-62 within the range of 80% to 125% of about 56 ng/mL at a median of about 1 hour following repeated administration of said dosage form every 12 hours through steady state conditions, whereby an arithmetic mean AUC_{0-inf} of T-62 is within the range of 80% to 125% of about 407 ng·h/mL.

38. An oral dosage form according to claims 37, wherein the carrier medium comprises at least one lipid excipient.

39. An oral dosage form according to claim 38, wherein the carrier medium further comprises one or more surfactants.

40. An oral dosage form according to claims 39, wherein the carrier medium is self-microemulsifying.

41. An oral dosage form according to claim 40, wherein the lipid excipient is soybean oil.

42. An oral dosage form according to claim 41, wherein the one or more surfactants are selected from the group consisting of propylene glycol monocaprylate, caprylocaproyl macrogol-8 glycerides and polysorbate 80.
